(19) United States
(12) Patent Application Publication
(10) Pub. No.: US 2024/0299504 A1
(43) Pub. Date: Sep. 12, 2024

(54) COMPOSITIONS AND METHODS FOR TREATING ALZHEIMERS DISEASE

(71) Applicants: CASE WESTERN RESERVE UNIVERSITY, Cleveland, OH (US); The United State Government as Represented by the Department of Veteran Affairs, Washington, DC (US)

(72) Inventors: Wen-Cheng Xiong, Shaker Heights, OH (US); Lin Mei, Shaker Heights, OH (US); Li Chen, Jilin (CN); Lei Xiong, Solon, OH (US)

(21) Appl. No.: 18/596,744

(22) Filed: Mar. 6, 2024

Related U.S. Application Data
(60) Provisional application No. 63/489,566, filed on Mar. 10, 2023.

(51) Int. Cl. A61K 38/29; A61B 6/50; A61P 25/28

(52) U.S. Cl. CPC A61K 38/29; A61B 6/505; A61P 25/28

(57) ABSTRACT

A method of treating Alzheimer's disease (AD) in a subject includes administering to the subject a therapeutically effective amount of at least one parathyroid hormone type 1 receptor (PTH1R) agonist.

### CROSS-REFERENCE TO RELATED APPLICATIONS

This application claims priority from U.S. Provisional Application No. 63/489,566, filed Mar. 10, 2023, the subject matter of which is incorporated herein by reference in its entirety.

### BACKGROUND OF THE INVENTION

Alzheimer's disease (AD) is the most common neurodegenerative disease that affects millions of people worldwide. Clinically, it is characterized by progressive cognitive deterioration accompanied by multisystem dysfunction. Pathologically, it features with increased β-amyloid (Aβ) plaques, hyperphosphorylated Tau+ neurofibrillary tangles, glial cell activation, brain inflammation, synaptic dysfunction, and neuron loss. Interestingly, in addition to brain pathologies, AD patients often have osteopenia or osteoporosis, a condition characterized by the loss of bone mass or bone mineral density (BMD) with deterioration of bone tissue micro-architectural, and a high incidence of hip fractures. β-amyloid peptide and phosphorylated tau have been detected in tissues outside of the central nervous system, including osteoporotic bone tissue, and they are negatively correlated with BMD. TgAPP_{swe}^{OCN} mice, that selectively express Swedish mutant APP (APP_{swe}) (a risk gene for early onset AD) in osteoblast lineage cells, exhibit not only bone-loss but also AD-associated brain pathological and behavioral phenotypes, suggesting a contribution of APP_{swe} induced bone deficits to the AD relevant brain pathology. However, the exact relationship between AD and osteoporosis remains elusive as it has yet to be demonstrated whether bone deficits contribute to AD development.

Parathyroid hormone (PTH) is a peptide secreted by the parathyroid glands that regulate serum calcium levels through its effects on bone, kidneys, and intestines. PTH analogs, such as PTH₁₋₃₄, are considered to be the most effective and currently FDA-approved anabolic therapy for patients with osteoporosis. PTH₁₋₃₄'s effects on bone depend primarily on the mode of its treatment. Chronically sustained treatment of PTH₁₋₃₄, acting as hyperparathyroidism, indirectly activates osteoclasts to exert a catabolic effect on bone mass, while intermittent PTH₁₋₃₄ administration activates osteoblasts to a large extent, increasing bone formation and thus bone mass.

### SUMMARY

Embodiments described herein relate to compositions and methods of treating Alzheimer's disease (AD) in a subject as well as methods of detecting Alzheimer's disease in a subject. Using a mouse model of AD that displays early onset β-amyloid (Aβ) based brain pathology and cognitive deficit, we demonstrated that a parathyroid hormone type 1 receptor (PTH1R) agonist, such as a PTH₁₋₃₄, is effective for inhibiting AD relevant brain pathology and improving cognitive function. Using the same mouse model, we further found that a PTH1R agonist, such as PTH₁₋₃₄, functions not only on osteoblasts in the bone, but also on astrocytes in the brain to suppress astrocyte senescence, activation, and the expression of inflammatory cytokines.

Accordingly, in some embodiments, a method of treating Alzheimer's disease in a subject includes administering to the subject a therapeutically effective amount of at least one PTH1R agonist, parathyroid hormone (PTH), or PTH analog.

In some embodiments, the PTH1R agonist can be administered at an amount effective to increase cognitive function and/or inhibit one or more AD brain pathologies in the subject. The one or more AD brain pathologies can be selected from astrocyte senescence, glial cell activation, expression of brain inflammatory cytokines, systemic inflammation, dystrophic neurites, Aβ accumulation, or Aβ deposition.

In some embodiments, the PTH1R agonist is administered at an amount effective to increase bone mass, bone density, bone thickness, and bone formation, or decrease bone resorption in the subject.

In some embodiments, the PTH1R agonist is intermittently administered to the subject. In some embodiments, the PTH1R agonist includes a PTH analog, such as PTH₁₋₃₄ peptide. In some embodiments, the PTH1R agonist is administered to the subject via subcutaneous or intravenous administration.

In some embodiments, the method can further include the step of measuring the bone mineral density (BMD) or bone thickness of the subject's skull. A reduced or decreased measured BMD or bone thickness in the subject's skull compared to a control is indicative of the subject having or having an increased risk of Alzheimer's disease. In some embodiments, the step of measuring the BMD in the subject's skull includes using a computed tomography (CT) or X-ray modality.

Other embodiments described herein relate to a method of treating Alzheimer's disease in a subject by measuring BMD or bone thickness of the skull of the subject and administering to the subject a therapeutically effective amount of at least one PTH1R agonist if the measured is BMD or bone thickness is reduced compared to a control BMD or bone thickness.

In some embodiments, the PTH1R agonist is administered at an amount effective to increase cognitive function and/or inhibit one or more AD brain pathologies in the subject. The one or more AD brain pathologies can be selected from the group consisting of astrocyte senescence, glial cell activation, expression of brain inflammatory cytokines, systemic inflammation, dystrophic neurites, Aβ accumulation and Aβ deposition. In some embodiments, the PTH1R agonist is administered at an amount effective to increase bone mass, bone density, bone thickness, and bone formation, or decrease bone resorption in the subject.

In some embodiments, the PTH1R agonist is intermittently administered to the subject. In some embodiments, the PTH1R agonist includes a PTH analog, such as a PTH₁₋₃₄ peptide. In some embodiments, the PTH1R agonist is administered to the subject via subcutaneous or intravenous administration. In some embodiments, the step of measuring the BMD in the subject's skull includes using a computed tomography (CT) or X-ray modality.

Still other embodiments described herein relate to a method of diagnosing Alzheimer's disease in a subject. The method includes measuring the bone marrow density (BMD) or bone thickness in a subject's skull. In some embodiments, a reduced BMD or bone thickness compared to a control is indicative of the subject having Alzheimer's disease or having an increased risk of Alzheimer's disease. In some embodiments, the step of measuring the BMD or bone thickness in the subject's skull includes using a computed tomography (CT) or X-ray modality.

### BRIEF DESCRIPTION OF DRAWINGS

FIGS. 1(A-H) illustrate Trabecular bone loss in 5×FAD mice was diminished by PTH1-34 treatments. A Schematic of hPTH₁₋₃₄ or vehicle (0.9% sodium chloride) intermittent treatment and tissue samples collection in WT/5×FAD female mice. Female WT/5×FAD mice were administered a once-daily injection of hPTH₁₋₃₄, or vehicle (0.9% NaCl) via subcutaneous injection, starting at 2˜MO old, 5 days per week. Mice were sacrificed at 6˜MO old to detect bone phenotypes. B-F μCT analysis of femurs from 6˜MO WT and 5×FAD female mice with PTH₁₋₃₄ or Veh treatment. Representative images are shown in B and quantification analyses of trabecular bone volume over total volume (Tb. BV/TV), trabecular bone number (Tb. N), trabecular bone thickness (Tb. Th), and cortical bone volume over total volume (Cb. BV/TV) by the direct model of CT analysis are presented in B-F. G Serum osteocalcin levels analyses, measured by ELISA assays, in 6˜MO female mice. H Serum PYD levels analyses, measured by ELISA assays, in 6˜MO female mice. Three different female mice from each group were examined. The data were presented as mean±SD, *P<0.05, **P<0.01, ***P<0.001, two-way ANOVA with two-stage step-up method where multiple comparisons test was used.

FIGS. 2(A-H) illustrate PTH₁₋₃₄ attenuation of cognition decline and memory loss in 5×FAD female mice. A Illustration of PTH₁₋₃₄ or vehicle intermittent treatment in WT/5×FAD female mice and behavioral testing schedule. All mice were tested for behavior from the age of 5˜MO old and treated with PTH₁₋₃₄/Veh continuously during the testing procedure. The results shown in this figure were for female mice. B, C Novel object recognition (NOR): Representative tracing images (B), and the quantification of discrimination index ([novel object explore time−old object explore time]/[total explore time]) of NOR (C) were shown. D Y maze: Quantifications of the total arm entries and spontaneous alternation. E-H Morris water maze (MWM): The latencies to reach the hidden platform during the training period (E), the representative tracing images (F), quantification of time spent in the target quadrant (G), and target zone crossovers (H) on the testing day were showed. All quantification data were presented as mean±SD (n=6-8 female mice per group). *P<0.05, **P<0.01, two-way ANOVA with two-stage step-up method where multiple comparisons test was used

FIGS. 3(A-L) illustrate PTH1-34 reduction of soluble AP level and in-soluble Aβ deposition in 5×FAD brain. A Illustration of PTH₁₋₃₄ intermittent treatment in 5×FAD mice. Mice were sacrificed at 5˜MO old to detect AP level and accumulation. B, C ELISA analyses of human Aβ40(B) and Aβ42(C) levels in the soluble fraction of brain homogenates including cortex and hippocampus (200 μg total protein) from female 5×FAD mice with PTH1-34 or Veh treatment (n=4 mice per group). D, E Representative images of ThioS staining for Aβ plaque depositions analysis in the cortex and hippocampus of 5×FAD-Veh (control) and 5×FAD-PTH1-34 mice. Representative images of female mice were shown in D and representative images of male mice were shown in E. F, G Quantification of plaque density (the amount of plaque deposition in each sub-region) in the cortex of 5×FAD female and male mice. H Quantification of the total plaque density in the cortex of female and male 5×FAD mice. I, J Quantification of plaque density in subregions of hippocampus in 5×FAD female and male mice. K Quantification of the total plaque density in the hippocampus of female and male 5×FAD mice. L Quantification of average plaque size in 5×FAD female and male mice. n=8 per group for female mice and n=6 per group for male mice in F-L. Scale bars as indicated in each panel. All data were presented as mean±SD. *P<0.05, **P<0.01, ***P<0.001, two-way ANOVA with Sidak's multiple comparisons test was used.

FIGS. 4(A-F) illustrate PTH₁₋₃₄ diminishment of GFAP+ reactive astrocytes. A Representative overall images of co-immunostaining with ThioS (green) and GFAP (red) of cortex and hippocampus from 6˜MO 5×FAD-Veh and 5×FAD-PTH₁₋₃₄ female mice. B Quantification of relative GFAP fluorescence intensity in each layer of cortex and subregional of hippocampus. C Representative images and high-magnification images in Aβ deposition regions of co-immunostaining with ThioS (green) and GFAP (red) of cortex and hippocampus from 6˜MO 5×FAD-Veh and 5×FAD-PTH₁₋₃₄ female mice. D Quantification of Abeta-associated GFAP fluorescence intensity. The Abeta-associated GFAP fluorescence intensity was defined by the intensity of GFAP-positive astrocytes in a plaque-centered circle within 50 m in diameter (marked by dashed white circles). n=8 mice per group. E Representative Western blots using antibodies against IBA1 and GFAP in homogenates of cortex and hippocampus from 6˜MO WT and 5×FAD female mice with PTH1-34 or Veh treatment. GAPDH was used as a loading control. F Quantification of relative protein level in E (n=5 mice per group). All quantification data were presented as mean±SD. Scale bars were indicated in each panel. *P<0.05, **P<0.01, ***P<0.001, two-way ANOVA with Sidak's multiple comparisons test was used.

FIGS. 5(A-F) illustrate PTH₁₋₃₄ diminishment of brain inflammation in 5×FAD mice. A Real-time PCR (RT-PCR) analysis of indicated gene expressions in the cortex of 6 MO WT and 5×FAD female mice with PTH₁₋₃₄ or Veh treatment. The level of GAPDH was normalized to 1, n=3 per group. B Total quantification of relative gene expression levels in A. The level of WT group was normalized to 1, n=10 gene per group. C The summary of altered genes in the 5×FAD-PTH₁₋₃₄ treatment group compared with the 5×FAD-Veh group in cortex. D RT-PCR analysis of indicated gene expressions in the hippocampus of 6˜MO female mice, n=3 per group. E Total quantification of relative gene expression levels in D. F The summary of altered genes in the 5×FAD-PTH₁₋₃₄ treatment group compared with the 5×FAD-Veh group in hippocampus. All quantification data were presented as mean±SD. *P<0.05, **P<0.01, ***P<0.001, one-way ANOVA with Tukey's multiple-comparison test was used.

FIGS. 6(A-F) illustrate PTH₁₋₃₄ reduction of serum inflammatory cytokines. A Representative images of serum L-Series label-multiplex antibody arrays of 6˜MO WT and 5×FAD female mice with PTH1-34 or Veh treatment. Proteins with changes are marked by dashed blue circles. B Total quantification analyses of the data in A. WT-Veh group were normalized to 1, n=36 proteins per group. C Quantification of relative serum protein levels in A. The data showed those proteins that were significantly changed and arranged into different groups according to their characteristics. The data were presented as mean±SD (n=4 mice per group), *P<0.05, **P<0.01, ***P<0.001, one-way ANOVA with Tukey's multiple-comparison test. D Heat map of serum protein. n=4, significant difference was set at P<0.05. E Comparison of the PTH₁₋₃₄ downregulated cytokines in serum of 5×FAD mice (downregulated cytokines in serum of PTH₁₋₃₄ treated 5×FAD mice over 5×FAD control mice) to those detected in the cortex or hippocampus of 5×FAD-PTH₁₋₃₄ mice. F Comparison between the 5×FAD with Tg2576 antibody array of secreted proteins in serum.

FIGS. 7(A-D) illustrate more PTH₁₋₃₄ association with astrocytes in 5×FAD brain than that of WT control. A Schematic diagram of experimental design. 2.5˜MO WT and 5×FAD female mice were administered PTH1-34-Biotin (100 μg/100 μl) or vehicle (phosphate buffer, 100 μl) by tail-intravenous injection to detect PTH₁₋₃₄ diffusion in vivo. Mice were sacrificed after 30 min for brain isolation and tissue sectioning, and immunofluorescence staining was used to analyze the distribution of PTH₁₋₃₄-Biotin. B Representative images and high-magnification images of Biotin (green) co-immunostaining with GFAP, IBA1, SLC16A1, and MAP2 (red) respectively from 5×FAD+Veh, 5×FAD+PTH1-34-Biotin and WT+PTH₁₋₃₄-Biotin female mice. All images were obtained from the cortex regions. Scale bars were indicated in the panel. C Quantification of relative Biotin fluorescence intensity in these three groups (mean±SD; n=8 mice per group). **P<0.01, ***P<0.001, one-way ANOVA with Kruskal-Wallis multiple-comparison test. D Quantification of PTH₁₋₃₄-Biotin distribution in 5×FAD brain cells from B. The distribution of PTH₁₋₃₄ in various cells was shown as a percentage, with a total proportion of 100%, derived from the mean value of data collected from all mice in the 5×FAD+PTH₁₋₃₄-Biotin group.

FIGS. 8(A-H) illustrate PTH1-34 decrement of senescence-like astrocytes from 5×FAD mice. A RT-PCR analysis of senescence-related gene expression level in the cultured astrocytes with PTH₁₋₃₄ or Veh treatment from WT and 5×FAD P3 female pups. GAPDH expression level was normalized to 1, n=3 independent experiments. B SA-β-gal staining of cultured WT and 5×FAD astrocytes with PTH₁₋₃₄ or Veh treatment. Scale bars as indicated in the panel. C Quantification of relative SA-β-gal+cell intensity in B (mean±SD; n=3 independent experiments). D RT-PCR analysis of senescence-related gene expression level in the hippocampus from 6˜MO WT or 5×FAD female mice with PTH1-34 or Veh treatment. GAPDH expression level was normalized to 1 (n=3). E Western blot analysis of indicated protein expression in homogenates of hippocampus of 6˜MO WT and 5×FAD female mice with PTH₁₋₃₄ or Veh treatment. F Quantification of relative protein level in E, n=4 mice per group. All quantification data were presented as mean±SD. *P<0.05, **P<0.01, one-way ANOVA with Tukey's multiple-comparison test was used. G Summary of AD brain pathology and cognitive function differences in 5×FAD mice with Veh or PTH₁₋₃₄ intermittent treatment. H Illustration of the working model.

FIGS. 9(A-B) illustrate decreased PTH in 5×FAD mice. (A) Serum PTH levels in 6-MO WT and 5×FAD female mice. (B) PTH levels in cortex and hippocampus of 6˜MO WT and 5×FAD female mice. The PTH levels were measured by Elisa. The values presented are means±SD (n=3 to 4). P values obtained by unpaired two-tailed t-test. * P<0.05, significant difference.

FIGS. 10(A-G) illustrate little to weak improvement of the cognitive functions in 5×FAD male mice by PTH₁₋₃₄. 5×FAD male mice with Veh or PTH1-34 treatment were subject to behavioral tests from 5˜MO of age, as was the case for 5×FAD female mice. The results shown in this figure were for male mice. (A, B) NOR: Representative tracing images (A), and quantification of the discrimination index of NOR (B) were shown. (C) Y maze: Quantifications of the total arm entries and spontaneous alternation in Y maze. (D-G) MWM: The latencies to reach the hidden platform during the training period (D), the representative tracing images (E), quantification of time in the target quadrant (F), and target zone crossovers (G) on the testing day. All quantification data were shown as mean±SD (n=7 mice for 5×FAD-Veh group and n=6 mice for 5×FAD-PTH1-34 group). *p<0.05, **p<0.01, two-way ANOVA with Sidak's multiple comparisons test was used in D, and Student's t-test was used in B, C, F, and G.

FIGS. 11(A-B) illustrate PTH₁₋₃₄ reduction of plague-associated dystrophic neurites. (A) Representative images of co-immunostaining with ThioS (green) and ATG9A (red) of the cortex and hippocampal sections from 6˜MO 5×FAD-Veh and 5×FAD-PTH₁₋₃₄ female mice. (B) Quantification of Dystrophic neurites (ATG9A+ positive area) in A. Scale bars were indicated in the panel. Quantification data were presented as mean±SD (n=8 for per group). *p<0.05, **p<0.01, ***p<0.001. Student's t-test.

FIGS. 12(A-G) illustrate little to no change in plaque-associated microglial cells in 5×FAD mice treated with PTH1-34. (A) Representative images of co-immunostaining with ThioS (green) and IBA1 (red) of cortex and hippocampus from 6˜MO 5×FAD-Veh and 5×FAD-PTH₁₋₃₄ female mice. (B) Quantification of the relative total IBA1 fluorescence intensity in A. (C) Quantification of Abeta un-associated IBA1 fluorescence intensity in A. The region used for quantification (no Aβ deposition area) were marked by white dashed squares, which were shown in A. (D) Representative images and high-magnification images in Aβ deposition regions of co-immunostaining with ThioS (green) and IBA1 (red) of cortex and hippocampus from 6-MO 5×FAD-Veh and 5×FAD-PTH₁₋₃₄ female mice. (E) Quantification of Abeta-associated IBA1 fluorescence intensity in D. The Abeta-associated IBA1 fluorescence intensity was defined by the intensity of IBA1 positive microglia in a plaque-centered circle within 50 m in diameter (marked by dashed white circles). n=8 mice per group. (F) Representative images of co-immunostaining with ThioS (white), IBA1 (green), and LPL (red) of cortical and hippocampal sections from 6-MO 5×FAD female mice with Veh/PTH1-34 treatments. (G) Quantification of Abeta-associated LPL+IBA1+/IBA1+ in F (n=6 mice per group). Scale bars as indicated in each panel. All quantification data were presented as mean±SD. *p<0.05, **p<0.01, ***p<0.001, Student's t-test.

FIGS. 13(A-D) illustrate significant reduction of glial cells in PTH₁₋₃₄ treated 5×FAD male mice. (A) Representative overall images and high-magnification images in the Aβ deposition region of co-immunostaining with ThioS (green) and GFAP (red) of cortex and hippocampus from 6˜MO 5×FAD-Veh and 5×FAD-PTH₁₋₃₄ male mice. (B) Quantification of total relative GFAP fluorescence intensity of cortex and hippocampus in A. (C) Representative overall images and high-magnification images in the Aβ deposition region of co-immunostaining with ThioS (green) and IBA1 (green) of cortex and hippocampus from 6˜MO 5×FAD-Veh and 5×FAD-PTH1-34 male mice. (D) Quantification of total relative IBA1 fluorescence intensity in C. Scale bars were indicated in each panel. All quantification data were presented as mean±SD (n=6 mice per group). *p<0.05, **p<0.01, ***p<0.001, Student's t-test was used.

FIGS. 14(A-D) illustrate little to no effect on astrocytes and microglia in the brains of WT mice treated with PTH1-34. (A) Representative images of co-immunostaining with ThioS (green) and GFAP (red) of cortex and hippocampus from 6˜MO WT-Veh and WT-PTH₁₋₃₄ female mice. (B) Quantification of relative GFAP fluorescence intensity and GFAP+ cell density in A. Cell density was defined by the number of positive cells per unit area (1 mm2). (C) Representative images of co-immunostaining with ThioS (green) and IBA1 (red) respectively of cortex and hippocampus from 6˜MO WT-Veh and WT-PTH₁₋₃₄ female mice. (D) Quantification of relative IBA1 fluorescence intensity and IBA1+ cell density in C. Scale bars were indicated in the panel. Quantification data were presented as mean±SD (n=4 for per group), Student's t-test.

FIGS. 15(A-D) illustrate PTH1R expression in astrocytes. (A) Representative Western blots using antibodies against PTH1R in homogenates of cultured astrocytes from WT and 5×FAD pups. β-Actin was used as a loading control. (B) Quantification of PTH1R protein level in A (mean±SD; n=5). *p<0.05, Student's t-test. (C) Representative images of RNA-Scope staining with PTH1R-mRNA, Glast-mRNA, IBA1 antibody, and DAPI of cortex sections from 6˜MO WT and 5×FAD female mice. High-magnification images, marked by dashed squares, were shown in the right panels. Scale bars as indicated in each panel. (D) Quantification analyses of PTH1R fluorescence distribution in C. The distribution of PTH1R in different cells was shown as a ratio, with a total ratio of 1. *p<0.05, mean±SD, n=6 mice per group, two-way ANOVA with Sidak's multiple comparisons test was used.

FIGS. 16(A-E) illustrate PTH₁₋₃₄ induction of signaling in astrocytes. (A) Illustration of astrocyte culture from WT or 5×FAD pups and the PTH1-34 treatment. Cells were collected at different times of treatment and different pathway proteins were detected. (B) Western blot analysis of indicated protein expression in cultured astrocytes with different treatment times. GAPDH was used as a loading control. (C-E) Quantification analyses of the data in B. The curves of p-CREB protein level (C), p-AKT protein level (D), and p-ERK protein level (E) over time are shown. The level of WT group with 0 min PTH₁₋₃₄ treatment was normalized to 1. The data were presented as mean±SD (n=3 independent experiments), *p<0.05, and two-way ANOVA with Tukey's multiple-comparison test was used.

FIGS. 17(A-F) illustrate PTH₁₋₃₄ suppression of proinflammatory cytokine expression in 5×FAD astrocytes. (A) Schematic of the experimental design. Primary astrocytes derived from the brains of P3 WT and 5×FAD pups were treated with PTH₁₋₃₄ or vehicle for 24 h for gene expression detection. (B) RT-PCR analysis of indicated gene expressions in the cultured astrocytes of four groups. The expression of GAPDH was normalized to 1, n=3 independent experiments, *p<0.05, **p<0.01, ***p<0.001, one-way ANOVA with Tukey's multiple-comparison test. (C) Total quantification of relative gene expression levels in B. The level of WT-Veh group was normalized to 1. *p<0.05, mean±SD, one-way ANOVA test. (D) Volcano plots analysis of altered gene expression level between 5×FAD and WT astrocytes. (E) Volcano plots analysis of 5×FAD astrocytes gene expression alteration in the PTH₁₋₃₄ treatment group. The red dots showed up-regulated genes and the blue dots showed down-regulated genes. (F) Comparison of the changes (down-regulated genes in PTH₁₃₄ treated 5×FAD astrocytes over 5×FAD control astrocytes) to those detected in the cortex or hippocampus of 5×FAD-PTH₁₋₃₄ mice.

### DETAILED DESCRIPTION OF THE INVENTION

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art.

For convenience, certain terms employed in the specification, examples, and appended claims are collected here. Unless defined otherwise, all technical and scientific terms used herein have the same meaning as commonly understood by one of ordinary skill in the art to which this application belongs.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.,”, as used herein are non-limiting and are for illustrative purposes only. “Including” and “including but not limited to” are used interchangeably.

The term “or” as used herein should be understood to mean “and/or” unless the context clearly indicates otherwise.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, 8%, ±7%, ±6%, ±5%, ±4%, ±3%, ±2%, or ±1% about a reference quantity, level, value, number, frequency, percentage, dimension, size, amount, weight or length.

As used herein, “one or more of a, b, and c” means a, b, c, ab, ac, be, or abc. The use of “or” herein is the inclusive or.

The term “administering” to a patient includes dispensing, delivering or applying an active compound in a pharmaceutical formulation to a subject by any suitable route for delivery of the active compound to the desired location in the subject (e.g., to thereby contact a desired cell), including administration into the cerebrospinal fluid or across the blood-brain barrier, delivery by either the parenteral or oral route, intramuscular injection, subcutaneous or intradermal injection, intravenous injection, buccal administration, transdermal delivery and administration by the rectal, colonic, vaginal, intranasal or respiratory tract route.

An “effective amount” of an agent or therapeutic peptide is an amount sufficient to achieve a desired therapeutic or pharmacological effect, such as an amount that is capable of increasing plasma levels of asprosin and/or the analogue thereof in a subject. An effective amount of an agent as defined herein may vary according to factors, such as the disease state, age, and weight of the subject, and the ability of the agent to elicit a desired response in the subject. Dosage regimens may be adjusted to provide the optimum therapeutic response. An effective amount is also one in which any toxic or detrimental effects of the active compound are outweighed by the therapeutically beneficial effects.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into a target tissue, such that it enters the animal's system and, thus, is subject to metabolism and other like processes, for example, subcutaneous administration.

The term “patient” or “subject” or “animal” or “host” or “individual” refers to any mammal. The subject may be a human but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

The terms “peptide” or “polypeptide” are used interchangeably herein and refer to compounds consisting of from about 2 to about 90 amino acid residues, inclusive, wherein the amino group of one amino acid is linked to the carboxyl group of another amino acid by a peptide bond. A peptide can be, for example, derived or removed from a native protein by enzymatic or chemical cleavage, or can be prepared using conventional peptide synthesis techniques (e.g., solid phase synthesis) or molecular biology techniques (see Sambrook et al., MOLECULAR CLONING: LAB. MANUAL (Cold Spring Harbor Press, Cold Spring Harbor, NY, 1989)). A “peptide” can comprise any suitable L- and/or D-amino acid, for example, common a-amino acids (e.g., alanine, glycine, valine), non-a-amino acids (e.g., P-alanine, 4-aminobutyric acid, 6aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and means for adding or removing protecting groups are known in the art. See, e.g., Green &amp; Wuts, PROTECTING GROUPS IN ORGANIC SYNTHESIS (John Wiley &amp; Sons, 1991). The functional groups of a peptide can also be derivatized (e.g., alkylated) using art-known methods.

Peptides can be synthesized and assembled into libraries comprising a few too many discrete molecular species. Such libraries can be prepared using well-known methods of combinatorial chemistry and can be screened as described herein or using other suitable methods to determine if the library comprises peptides which can sequester asprosin. Such peptides can then be isolated by suitable means.

The term “peptidomimetic”, refers to a protein-like molecule designed to mimic a peptide. Peptidomimetics typically arise either from modification of an existing peptide, or by designing similar systems that mimic peptides, such as peptoids and P-peptides. Irrespective of the approach, the altered chemical structure is designed to advantageously adjust the molecular properties such as, stability or biological activity. These modifications involve changes to the peptide that do not occur naturally (such as altered backbones and the incorporation of nonnatural amino acids).

The terms “portion”, “fragment”, “variant”, “derivative” and “analog” or “analogue”, when referring to a polypeptide include any polypeptide that retains at least some biological activity referred to herein (e.g., inhibition of an interaction such as binding). Polypeptides as described herein may include portion, fragment, variant, or derivative molecules without limitation, as long as the polypeptide still serves its function. Polypeptides or portions thereof of the present invention may include proteolytic fragments, deletion fragments and in particular, or fragments that more easily reach the site of action when delivered to an animal.

In some embodiments, a “portion” or “fragment” polypeptide (including a domain) will be understood to mean a polypeptide of reduced length (e.g., reduced by 1, 2, 3, 4, 5, 6, 7, 8, 9, 10, 11, 12, 13, 14, 15, 16, 17, 18, 19, 20 or more residue(s) (e.g., peptide(s)) relative to a reference polypeptide, respectively, and comprising, consisting essentially of and/or consisting of a polypeptide of contiguous residues, respectively, identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference polypeptide.

Different proteins having homology are referred to herein as “homologues.” The term homologue includes homologous sequences from the same and other species and orthologous sequences from the same and other species. “Homology” refers to the level of similarity between two or more amino acid sequences in terms of percent of positional identity (i.e., sequence similarity or identity). Homology also refers to the concept of similar functional properties among different proteins. Thus, the compositions and methods described herein further comprise homologues to the polypeptides of this invention. “Orthologous” and “orthologs” as used herein, refers to homologous amino acid sequences in different species that arose from a common ancestral gene during speciation. A homologue or ortholog of a amino acid sequence of this invention can have a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the amino acid sequence described herein.

The term “sequence identity” refers to the extent to which two optimally aligned polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

The term “percent identity” can refer to the percentage of identical amino acids in an amino acid sequence as compared to a reference polypeptide.

The phrase “substantially identical,” or “substantial identity” in the context of two polypeptide sequences, or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% amino acid residue identity, when compared and aligned for maximum correspondence, as measured using one of the following sequence comparison algorithms or by visual inspection. In some embodiments, the substantial identity exists over a region of consecutive amino acids of an amino acid sequence of the invention that is about 2 amino acids to about 20 amino acids, about 10 amino acids to about 25 amino acids, about 10 amino acids to about 30 amino acids, about 15 amino acids to about 25 amino acids, about 30 amino acids to about 40 amino acids, about 50 amino acids to about 60 amino acids, about 70 amino acids to about 80 amino acids, about 90 amino acids to about 100 amino acids, or more amino acids in length, and any range therein, up to the full length of the amino acid sequence. In some embodiments, the amino acids sequences can be substantially identical over at least about 20 amino acids (e.g., about 20, 21, 22, 23, 24, 25, 26, 27, 28, 29, 30, 31, 32, 33, 34, 35, 36, 37, 38, 39, 40 amino acids). In some embodiments, a substantially identical protein sequence performs substantially the same function as the protein sequence to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG. Wisconsin Package (Accelrys Inc., San Diego, Calif.). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100.

The term “linked,” or “fused” in reference to polypeptides, refers to the attachment of one polypeptide to another. A polypeptide may be linked or fused to another polypeptide (at the N-terminus or the C-terminus) directly (e.g., via a peptide bond) or through a linker (e.g., a peptide linker).

The term “linker” in reference to polypeptides is art-recognized and refers to a chemical group, or a molecule linking two molecules or moieties, e.g., two domains of a fusion polypeptide protein. A linker may be comprised of a single linking molecule (e.g., a single amino acid) or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide.

The term “therapeutically effective” means that the amount of the composition used is of sufficient quantity to ameliorate one or more causes, symptoms, or sequelae of a disease or disorder. Such amelioration only requires a reduction or alteration, not necessarily elimination, of the causes, symptoms, or sequelae of a disease or disorder.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The agents, compounds, compositions, polypeptides, proteins, etc. used in the methods described herein are considered to be purified and/or isolated prior to their use. Purified materials are typically “substantially pure”, meaning that a nucleic acid, polypeptide or fragment thereof, or other molecule has been separated from the components that naturally accompany it. Typically, the polypeptide is substantially pure when it is at least 60%, 70%, 80%, 90%, 95%, or even 99%, by weight, free from the proteins and other organic molecules with which it is associated naturally. For example, a substantially pure polypeptide may be obtained by extraction from a natural source, by expression of a recombinant nucleic acid in a cell that does not normally express that protein, or by chemical synthesis. “Isolated materials” have been removed from their natural location and environment. In the case of an isolated or purified domain or protein fragment, the domain or fragment is substantially free from amino acid sequences that flank the protein in the naturally-occurring sequence.

The term “cognitive function” refers to mental processes involved in the acquisition of knowledge, manipulation of information, and reasoning. Cognitive functions can include the domains of perception, memory, learning, attention, decision making, language abilities, awareness, judgement, and mental acuity.

The term “cognitive decline” refers to a reduction in one or more cognitive abilities or functions, such as perception, memory, learning, attention, decision making, language abilities, awareness, judgement, and mental acuity, across the adult lifespan. The presence and degree of decline varies with the cognitive ability being measured as fluid abilities often show greater declines than crystallized. Cognitive decline is a part of normal healthy aging, but a severe decline is not normative and could be symptomatic of disease. Cognitive decline is the primary symptom of disease-induced dementias, such as Alzheimer's disease.

The term “Alzheimer's disease” (AD) refers to an age-related, progressive brain disorder associated with general degeneration of the brain and initially manifesting itself with partial amnesia, and later restlessness, disorientation, aphasia, agnosia or apraxia (cognitive decline), dementia and sometimes euphoria or depressions. AD may be associated with neurological and communication disorders and the National Institute of Stroke Neurological and Communicative Disorders and Stroke, and Alzheimer's Disease and Related Disorders Association criteria.

Embodiments described herein relate to compositions and methods of treating Alzheimer's disease (AD) in a subject as well as methods of detecting Alzheimer's disease in a subject. Using a mouse model of AD that displays early onset β-amyloid (Aβ) based brain pathology and cognitive deficit, we demonstrated that a parathyroid hormone type 1 receptor (PTH1R) agonist, such as a PTH₁₋₃₄, is effective for inhibiting AD relevant brain pathology and improving cognitive function. Using the same mouse model, we further found that a PTH1R agonist, such as PTH₁₋₃₄, functions not only on osteoblasts in the bone, but also on astrocytes in the brain to suppress astrocyte senescence, activation, and the expression of inflammatory cytokines.

Accordingly, in some embodiments, a method of treating Alzheimer's disease in a subject includes administering to the subject a therapeutically effective amount of at least one PTH1R agonist, PTH, or PTH analog.

In some embodiments, the PTH1R agonist can include any agent that can enhance, increase, or promote one or more of PTH1R activity, PTH1R function, or PTH1R signaling. The activity, signaling, and/or function of PTH1R can be enhanced, increased and/or promoted in several ways including: direct enhancement of the activity of the PTH1R (e.g., by using small molecules and/or peptide agonists); activation of genes and/or proteins that enhance, increase, and/or promote one or more of, the activity, signaling, and/or function of the PTH1R (e.g., by increasing the expression or activity of the genes and/or proteins); promotion of genes and/or proteins that are downstream mediators of the PTH1R activity (e.g., by enhancing the expression and/or activity of the mediator genes and/or proteins); introduction of genes and/or proteins that positively regulate one or more of, activity, signaling, and/or function of PTH1R (e.g., by using recombinant gene expression vectors, recombinant viral vectors or recombinant polypeptides); or gene replacement with, for instance, a hypermorphic mutant of the PTH1R (e.g., by homologous recombination, overexpression using recombinant gene expression or viral vectors, or mutagenesis).

In some embodiment, the PTH1R agonist that enhances, increases, and/or promotes one or more of the activity, signaling, and/or function of the PTH1R, can include a therapeutic polypeptide or small molecule that binds to and/or complexes PTH1R to enhance, increase or promote the activity, signaling, and/or function of PTH1R.

In some embodiments, the therapeutic polypeptides or small molecules that act as a PTH1R agonist can inhibit amyloid beta (Aβ)-associated brain pathologies and improve cognitive function in subjects having and/or having an increased risk of AD.

In some embodiments, the PTH1R agonist is a therapeutic polypeptide or protein that when introduced into the circulation of a subject in need thereof can bind and/or complex to the membrane bound PTH1R, a G-protein coupled receptor (GPCR), to induce PTH1R activity, signaling, and/or function in cells, such as astrocytes in the brain and osteoblasts in bone. For example, PTH1R signaling is mediated through both the Gs and Gq G-proteins as well as through P-arrestin in a G-protein independent manner.

PTH1R has two native agonists, parathyroid hormone (PTH) and parathyroid hormone-related protein (PTHrP). Therefore, in some embodiments, the PTH1R agonist can include a recombinant PTH, such as rhPTH₁₋₈₄, a PTH analog, such as rhPTH₁₋₃₄ or PTH₁₋₃₄, recombinant PTHrP or a recombinant PTHrP analog, such as rhPTHrP₁₋₃₄.

A PTH polypeptide for use as a PTH1R agonist in accordance with a method described herein can include PTH polypeptides from mammalian species, more preferably from human and murine species, as well as their variants, analogs, orthologs, homologs, and derivatives and fragments thereof that are characterized by binding to and activating the PTH1R receptor. Exemplary PTH polypeptides can include at least one PTH polypeptide selected from the group consisting of amino acid SEQ ID NOs: 1-60 as disclosed in US 2023/0121525 A1 which are herein incorporated by reference in their entirety.

In some embodiments, the PTH1R agonist can include a PTH analog. In some embodiments the PTH1R agonist can include PTH₁₋₃₄ (also known as Teriparatide). PTH₁₋₃₄ is an FDA approved recombinant human PTH analog that includes the first (N-terminus) 34 amino acids corresponding to the bioactive portion of PTH.

In some embodiments, the PTH1R agonist can include a PTHrP polypeptide, that binds to and activate common PTH/PTHrP1 receptor, commonly known as PTHR1. In some embodiments, the PTH1R agonist can include a PTHrP analog. In some embodiments, the PTHrP analog can include an analog of PTHrP₁₋₃₄, such as Abaloparatide (ABL). ABL is an FDA approved 34 amino acid synthetic analog of PTHrP₁₋₃₄ that is identical to PTHrP₁₋₃₄ at amino acid residues 1-22, but with multiple substitutions between amino acids 23-34 (38% identical to PTHrP) to maximize the stability of the molecule. Abaloparatide preferentially binds the RG conformational state of the PTH1R, which in turn elicits a transient downstream cyclic AMP signaling response towards to a more anabolic signaling pathway. In some embodiments, the PTH1R agonist can include PTHrP₁₋₃₆. PTHrP₁₋₃₆ is a recombinant form of a mature, N-terminal secretory peptide derived from a PTHrP preprohormone.

The PTH1R agonist can include a hybrid protein. In some embodiments, a PTH1R agonist can include a hybrid protein derived from PTH and PTHrP. A hybrid protein derived from PTH and PTHrP can include “long-acting PTH” (LA-PTH). LA-PTH which is a long-acting hybrid of PTH and PTH related peptide. (PTH(1-14)/PTHrP(15-36) ([Ala^{1,3,12},Gln¹⁰,Arg¹¹,Trp¹⁴]PTH(1-14)/[Ala18,22,Lys26]PTHrP(15-36)COOH)). In some embodiments the hybrid protein can include a PTH polypeptide and a collagen binding domain as described by Ponnapakkam et al. (Drug Discov Today, 2014, 19(3), 204, 208), which are herein incorporated by reference in their entirety. In some embodiments, a PTH polypeptide can be conjugated, directly or via a linker, to albumin, or a domain of albumin, as described in U.S. Pat. No. 7,592,010, which are herein incorporated by reference in their entirety.

In some embodiments, the PTH1R agonist has a binding affinity KD to PTH1R less than about 10 μM, less than about 1 μM, less than about 500 nM, less than about 400 nM, less than about 300 nM, less than about 200 nM, less than about 100 nM, less than about 10 nM, less about 1 nM, or less than about 500 pM.

A PTH1R agonist described herein can be subject to other various changes, substitutions, insertions, and deletions where such changes provide for certain advantages in its use. In this regard, a PTH1R agonist that has an amino acid sequence substantially identical to PTH and/or PTHrP that binds to PTH1R can correspond to or be substantially homologous with, rather than be identical to, the sequence of a recited polypeptide where one or more changes are made and it retains the ability to increase or promote one or more of activity, signaling, and/or function of PTH1R.

The PTH1R agonist can be in any of a variety of forms of polypeptide derivatives that include amides, conjugates with proteins, cyclized polypeptides, polymerized polypeptides, analogs, homologs, orthologs, fragments, chemically modified polypeptides and the like derivatives.

The PTH1R agonist can also include conservative substitutions of amino acid residues. It will be appreciated that the conservative substitution can also include the use of a chemically derivatized residue in place of a non-derivatized residue provided that such peptide displays the requisite binding activity.

“Chemical derivative” refers to a subject peptide having one or more residues chemically derivatized by reaction of a functional side group. Such derivatized molecules include for example, those molecules in which free amino groups have been derivatized to form amine hydrochlorides, p toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those polypeptides, which contain one or more naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine; and ornithine may be substituted for lysine. Polypeptides described herein may also include any polypeptide having one or more additions and/or deletions or residues relative to the sequence of a polypeptide whose sequence is shown herein, so long as the requisite activity is maintained.

In some embodiments, one or more of the PTH1R agonist polypeptides described herein can be included in a PTH1R agonist polypeptide prodrug. A prodrug of a PTH1R agonist polypeptide described herein can preferably exhibit low residual activity of the prodrug while providing a sustained release of the PTH1R agonist. Suitable PTH and PTHrP polypeptide prodrugs are known in the art, such as for example the PTH and PTHrP prodrugs including a PTH moiety, a reversible prodrug linker moiety, a chemical bond or spacer moiety, and a water-soluble carrier moiety, as described in US 2023/0121525 A1 which are herein incorporated by reference.

One or more of the PTH1R agonist polypeptides described herein can also be modified by natural processes, such as posttranslational processing, and/or by chemical modification techniques, which are known in the art. Modifications may occur in the peptide including the peptide backbone, the amino acid sidechains and the amino or carboxy termini. It will be appreciated that the same type of modification may be present in the same or varying degrees at several sites in a given peptide. Modifications comprise for example, without limitation, acetylation, acylation, addition of acetomidomethyl (Acm) group, ADP-ribosylation, amidation, covalent attachment to flavin, covalent attachment to a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination (for reference see, Protein-structure and molecular properties, 2nd Ed., T. E. Creighton, W. H. Freeman and Company, New-York, 1993).

In some embodiments, one or more PTH1R agonist polypeptide can include an amide derivative. Exemplary PTH polypeptide and PTHrP polypeptide amide derivatives wherein the C-terminus is amidated can be selected from the group consisting of amino acid SEQ ID NOs: 61-121 as disclosed in US 2023/0121525 A1 which are herein incorporated by reference in their entirety.

Peptides, polypeptides, and/or proteins described herein may also include, for example, biologically active mutants, variants, fragments, chimeras, and analogues; fragments encompass amino acid sequences having truncations of one or more amino acids, wherein the truncation may originate from the amino terminus (N-terminus), carboxy terminus (C-terminus), or from the interior of the protein. Analogs described herein can involve an insertion or a substitution of one or more amino acids.

One or more of the PTH1R agonist polypeptides described herein can also encapsulated in a nano- or microparticle or conjugated to either synthetic or peptidic polymers. In some embodiments, a PTHR1 agonist polypeptide can be encapsulated in a poly lactic-co-glycolic acid (PLGA) biodegradable polymer to form a longer lasting version of the PTH1R agonist polypeptide.

In some embodiments, a PTH1R agonist polypeptide can include a recombinant PTH1R agonist-Fc fusion protein to extend the half-life of the PTH1R agonist polypeptide. “Fc,” as used herein, refers to the Fc region of a human IgG antibody. Fc-Fusion proteins (also known as Fc chimeric fusion protein, Fc-Ig, Ig-based Chimeric Fusion protein and Fc-tag protein) are composed of the Fc domain of IgG chemically linked to a peptide or protein of interest (e.g., PTH). Fe domains from any IgG may be used. By way of a non-limiting example, Fe domains of IgG1, or IgG2, or IgG3, or IgG4 may be used, as well as various combinations of Fe domains originating from different IgGs, such as half IgG2 and half IgG4. In one non-limiting embodiment, PTH1R agonist-Fc fusion proteins of the invention have Fe domains of a human IgG1 antibody. The PTH1R agonist sequence may be directly or indirectly linked to an Fc domain. In some embodiments, a PTH1R agonist is linked to an Fc domain directly. In other embodiments, PTH1R agonist is linked to an Fc domain by a linker, such as an amino acid linker.

In some embodiments, the PTH1R agonist-Fc fusion protein can include a human PTH₁₋₃₄ joined at its C-terminus to the Fc fragment of human IgG1 (PTH-FC) as described by Kostenuik et al. (J Bone Miner Res, 2007, 22(1)), 1534-1547), which are herein incorporated by reference in their entirety. Additional PTH-Fc fusion protein variants for use as a PTH1R agonist in compositions and methods described herein can include a PTH polypeptide, or a variant thereof, chemically linked to an Fc region of a human IgG antibody, or a derivative thereof as described in US 2020/0247865 A1, which are herein incorporated by reference in their entirety.

PTH1R agonist polypeptides described herein may be prepared by methods known to those skilled in the art. The PTH1R agonist polypeptides may be prepared using recombinant DNA. For example, one preparation can include cultivating a host cell (bacterial or eukaryotic) under conditions, which provide for the expression of PTH1R polypeptide agonist within the cell.

The purification of the PTH1R agonist polypeptide may be done by affinity methods, ion exchange chromatography, size exclusion chromatography, hydrophobicity or other purification technique typically used for protein purification. The purification step can be performed under non-denaturing conditions. On the other hand, if a denaturing step is required, the polypeptide or protein may be renatured using techniques known in the art.

In some embodiments, the PTH1R agonist is an exogenous peptide that can be recombinantly produced and systemically administered to the subject by, for example, parenteral or intravenous administration.

In some embodiments, the PTH1R agonist can include a small molecule agent. In some embodiments, the small molecule PTH1R agonist can include the hPTH1R agonist PCO371 and derivatives thereof. AnCPO371 derivative can include the orally active PTH1R agonist 1-(3,5-dimethyl-4-(2-((4-oxo-2-(4-(trifluoromethoxy)phenyl)-1,3,8-triazaspiro[4.5]dec-1-en-8-yl)sulfonyl)ethyl)phenyl)-5,5-dimethylimidazolidine-2,4-dione (PCO371,16c)

The PTH1R agonists described herein may be formulated with one or more pharmaceutically acceptable carrier or excipients to provide a pharmaceutical composition. The PTH1R agonists described herein may be combined with a pharmaceutically acceptable buffer, and the pH adjusted to provide acceptable stability, and a pH acceptable for administration such as parenteral administration. Optionally, one or more pharmaceutically acceptable anti-microbial agents may be added. Meta-cresol and phenol are preferred pharmaceutically acceptable microbial agents. One or more pharmaceutically acceptable salts may be added to adjust the ionic strength or tonicity. One or more excipients may be added to further adjust the isotonicity of the formulation. Glycerin is an example of an isotonicity-adjusting excipient. Pharmaceutically acceptable means suitable for administration to a human or other animal does not contain toxic elements or undesirable contaminants and does not interfere with the activity of the active compounds therein.

The PTH1R agonists can be delivered to a subject by any suitable route, including, for example, local and/or systemic administration. Systemic administration can include, for example, parenteral administration, such as intramuscular, intravenous, intraarticular, intraarterial, intrathecal, subcutaneous, or intraperitoneal administration. In some embodiments, the PTH1R agonists can be delivered to a subject via subcutaneous administration. The PTH1R agonists can also be administered orally, transdermally, topically, by inhalation (e.g., intrabronchial, intranasal, oral inhalation or intranasal drops) or rectally. In some embodiments, the PTH1R agonists can be administered to the subject via intravenous administration using an infusion pump to deliver daily, weekly, or doses of the therapeutic agent.

Pharmaceutically acceptable formulations of the PTH1R agonists described herein can be suspended in aqueous vehicles and introduced through conventional hypodermic needles or using infusion pumps.

For injection, PTH1R described herein can be formulated in liquid solutions, typically in physiologically compatible buffers such as Hank's solution or Ringer's solution. In addition, the PTH1R agonists may be formulated in solid form and re-dissolved or suspended immediately prior to use. Lyophilized forms are also included. The injection can be, for example, in the form of a bolus injection or continuous infusion (such as using infusion pumps) of the therapeutic agent.

It will be appreciated that the amount, volume, concentration, and/or dosage of the PTH1R agonists that is administered to any one animal or human depends on many factors, including the subject's size, body surface area, age, the particular composition to be administered, sex, time and route of administration, general health, and other drugs being administered concurrently. Specific variations of the above noted amounts, volumes, concentrations, and/or dosages of the PTH1R agonists can readily be determined by one skilled in the art using the experimental methods described below.

In some embodiments, an effective amount of one or more PTH1R agonists described herein can range from about 0.01 μg/Kg to about 5 or 10 mg/kg, inclusive; administered daily, weekly, biweekly, monthly, or less frequently. In some embodiments, an effective amount of one or more PTH1R agonists described herein can range from about 1 μg/Kg to about 500 μg/kg administered daily, or about 5 μg/Kg to about 100 μg/kg administered daily, or about 10 μg/Kg to about 80 μg/kg administered daily, or about 50 μg/kg administered daily. In an exemplary embodiment, the effective amount of PTH₁₋₃₄ is about 50 μg/Kg administered daily.

In some embodiments, one or more PTH1R agonists can be intermittently administered to the subject. Intermittent administration can include a treatment cycle followed by a non-treatment interval. Therefore, in some embodiments, an amount of PTH1R agonist can be administered to a subject over a set period of time either as a one-off dose or at repeated time intervals and then stopped until the next dose is required.

In some embodiments, intermittently administration can include once daily administration of one or more PTH1R agonists to a subject for 2, 3, 4, 5, or 6 days/week. In some embodiments, the one or more PTH1R agonist can be administered to the subject once daily 5 days/week for at least 2-3 months.

Pharmaceutical compositions including a PTH1R agonists described herein, such as PTH₁₋₃₄ or an analog thereof, can be administered to any subject having or at an increased risk of Aβ deposition, aggregation, and/or accumulation in the brain that can experience the beneficial effects of increasing or promoting PTH1R signaling, PTH1R activity, and/or PTH1R function in the subject. Foremost among such animals are humans, although the present invention is not intended to be so limited.

The PTH1R agonists described herein can be used in methods and compositions for treating or preventing amyloid-beta (Aβ) associated brain pathologies in a subject. Aβ monomers are easily self-assembled into oligomers, protofibrils and beta-sheet-rich fibers, and are related to the pathogenesis of neurotoxicity. As used herein, the term “Aβ-associated brain pathologies” is used to encompass the development all diseases that may be caused by and/or related to the deposition, aggregation, and/or accumulation of Aβ in the brain. Examples of the Aβ-associated brain pathologies include, but are not limited to, dementia (e.g., Alzheimer's disease, vascular dementia, etc.), mild cognitive impairment, cerebral amyloid angiopathy, Down's syndrome, amyloid stroke, systemic amyloid bodies (DLB), multi-infarct dementia (MID), frontotemporal lobar degeneration (FTLD), Pick's disease, corticobasal degeneration (CBD), progressive supranuclear palsy (PSP), Parkinson's disease, Huntington's disease, and the like. Additional Aβ-associated brain pathologies treated in accordance with a method described herein can include, but are not limited to, Dutch amyloidosis, tauopathy, dementia with Lewy.

An important pathological feature of Alzheimer's disease (AD), which is a representative neurodegenerative disease, is the formation of peptide aggregates called “senile plaques”, which causes synaptic dysfunction and neuronal death. While Aβ is a normal peptide generated throughout life, Aβ is the main component in neuritic plaques that are a neuropathological hallmark of AD.

Therefore, in some embodiments, the pharmaceutical compositions described herein can be administered to a subject for the treatment of AD. Unambiguous diagnosis of AD requires clinical findings of cognitive deficits consistent with AD and post-mortem identification of brain pathologies consistent with AD. The term “probable Alzheimer's disease” is used when a subject demonstrates clinical characteristics of AD and when other possible biological causes of dementia (e.g. Parkinson's disease or stroke) are excluded.

There are a variety of art-accepted methods for diagnosing probable Alzheimer's disease including those described below. Typically, methods of diagnosing AD are used in combination. These methods include determining an individual's ability to carry out daily activities and identifying changes in behavior and personality. Dementia of the AD type is also typically characterized by an amnestic presentation (memory deficit) or language, visuospatial or executive function deficits.

Subject having AD can also suffer from a progressive decline in several forms of memory, including associative memory and/or novel object recognition. Therefore, in some embodiments, subjects to be treated for AD in accordance with a method described herein can also have cognitive decline and/or memory deficits. Memory deficits can include associative memory deficits and/or novel object recognition memory deficits. Associative memory can refer to the ability to learn and remember the relationship between unrelated items or concepts. Novel object recognition memory can include a subject's ability to recognize a novel object in the environment. In some embodiments, novel object recognition memory can be indicative of alterations in the working memory, attention, anxiety, and innate preference for novelty in a subject.

In some embodiments, at least one PTH1R agonist is administered to a subject for the treatment of AD at an amount effective to increase cognitive function and/or inhibit one or more AD brain pathologies in the subject. AD brain pathologies inhibited in accordance with a method described herein can include, but are not limited to, astrocyte senescence, glial cell activation, expression of brain inflammatory cytokines, brain inflammation, systemic inflammation, dystrophic neurites, AB accumulation and AB deposition.

In certain embodiments, at least one PTH1R agonist is administered at an amount effective to increase cognitive function in the subject. Cognitive function/ability/impairment/decline/improvement may be determined by art-accepted methods, including, but not limited to, validated instruments that assess global cognition (e.g., the Modified Mini Mental State Examination (3MS-E)), and specific domains such as visual and verbal memory (e.g., the Brief Visuospatial Memory Test (Revised) (BVMT-R) and the Hopkins Verbal Learning Test (Revised) (HVLT-R), respectively), language (e.g., the Generative Verbal Fluency Test (GVFT)) and executive function and attention (e.g., the Digit Span Test (DST)).

It was shown that a PTH1R agonist treatment reduced senescence markers (e.g., SA-β-Gal, and p53) in astrocytes in the AD mouse model. Therefore, in some embodiments, at least one PTH1R agonist can be administered to a subject at an amount effective to inhibit and/or reduce astrocyte senescence in the subject's brain.

PTH1R agonist treatment was shown in an AD mouse model to reduce glial cell activation and the consequent brain inflammation resulting from the expression of well recognized proinflammatory cytokines and chemokines (e.g., IL1β, IL6, TNFα, RANK, CCL5 or TGFβ1). Glial cell activation can include IBA1+ microglia and GFAP+ astrocyte activation. Therefore, in some embodiments, at least one PTH1R agonist can be administered at an amount effective to inhibit glial cell activation (e.g., microglia or astrocyte activation) and/or proinflammatory cytokine or chemokine (e.g., IL1β, IL6, TNFα, RANK, CCL5 or TGFβ1) expression in the subject's brain. Glial activation can be assessed using one or more of immunohistochemical staining analysis, western blots, and behavioral tests. In particular embodiments, at least one PTH1R agonist can be administered to a subject at an amount effective to inhibit brain inflammation in the subject, such as inflammation resulting from glial cell activation and/or proinflammatory cytokine or chemokine expression in the subject's brain.

PTH1R agonist treatment was also shown in an AD mouse model to reduce serum inflammatory cytokines (e.g., IL-1rα, IL-2, IL-3, IL-4, IL-10, CXCL9, CCL3) as well as serum inflammation-associated factors (e.g., IL1β, TNFα, TREM-1, CCL11, and CCL5) while restoring serum factor CCL4, which was decreased in AD model mouse. Therefore, in some embodiments, at least one PTH1R agonist can be administered to a subject at an amount effective to inhibit systemic inflammation in the subject. In addition, systemic and brain inflammation can be evaluated by serum cytokine array, real-time PCR (qPCR), and RNAscope.

Swollen, bulbous-shaped (dystrophic) neurites are a common pathologic feature of Alzheimer's disease (AD) and represent one of the most abundant neuritic abnormalities within the brains of patients with this disease. Therefore, in some embodiments, at least one PTH1R agonist can be administered to a subject at an amount effective to inhibit plaque associated dystrophic neurites in the subject's brain.

PTH1R agonist treatment was further shown in an AD mouse model to reduce soluble Aβ levels as well as in-soluble Aβ deposition in the brain. Therefore, in some embodiments, at least one PTH1R agonist can be administered to a subject at an amount effective to inhibit Aβ accumulation and/or Aβ deposition in the subject's brain.

The bone phenotypes of the AD mouse model and wild-type mice were examined using computed tomography (CT) and evaluated by measuring serum bone formation and resorption markers. It was found that AD-relevant mouse model mice demonstrate an increase in skeletal bone deficits compared to wild-type animals. Bone deficits found to be increased in mouse models of AD compared to wild type animals include reduced bone mass, a decrease in bone formation, a decrease in bone thickness, and an increase in bone resorption. Thus, it is contemplated that the presence of, or measured level of, one or more bone deficits in a subject can be indicative of a subject having or having an increased risk of Alzheimer's disease.

Therefore, other embodiments described herein relate to a method of diagnosing Alzheimer's disease in a subject. The method includes measuring the bone mineral density (BMD) or bone thickness in a subject's skull. In some embodiments, the measurement is taken from a superior portion of the frontal bone, occipital bone, and/or parietal bones of the calvaria, or top part of the skull. According to the present invention, a reduced or decreased BMD or bone thickness measurement compared to a control is indicative of the subject having or having an increased risk of Alzheimer's disease.

The term “measuring” is used according to its ordinary and plain meaning to refer to ascertaining or determining the amount, value, degree, size or level of something. In some embodiments a bone density test is used to measure bone mineral content and density in the subject's skull. In some embodiments, X-rays, dual-energy X-ray absorptiometry (DEXA or DXA), or a CT scan can be used to determine bone density or bone thickness of one or more portions of a subject's skull. In particular embodiments, the BMD or bone thickness of a subject's skull is measured by CT.

The measured BMD or bone thickness can be compared with a control value, control score, or control level to correlate the measured BMD or bone thickness to the subject having or having an increased risk of Alzheimer's disease. The control value, control score, or control level can be the measured BMD or bone thickness in a healthy subject (considered a normal BMD or bone thickness score), for example a healthy subject that does not have Alzheimer's disease or osteoporosis. In some embodiments, the control value, control score, or control level can be the measured BMD or bone thickness in a healthy subject having a similar age, ethnicity and/or sex as the subject. A subject whose BMD or bone thickness is less than a control BMD or bone thickness value, score or level found in a comparable healthy subject in a can be indicative of the subject having Alzheimer's disease or having an increased risk of Alzheimer's disease.

It is contemplated that one or more standards may be generated in which a control value or level is defined by a normal BMD or bone thickness value, score, or level. That standard may then be referred to as a way of determining whether BMD or bone thickness measured in a given subject's skull is normal or below-normal. The type of standard generated will depend upon the assay or test employed to evaluate BMD or bone thickness in the subject's skull. In some embodiments of the invention, a score is assigned to the subject's BMD or bone thickness based on certain criteria and numbers within or below a certain number or range are deemed “below normal.”

In some aspects of the invention, BMD or bone thickness in a subject's skull is considered below normal if an assay indicates that a particular measurement, amount, value, score, or level is at about or at least about 80%, 75%, 70%, 65%, 60%, 55%, 50%, 45%, 40%, 35%, 30%, 25%, 20%, 15%, 10%, 5% or less than the measurement, amount, value, score or level measured in subject's that have normal BMD or bone thickness, e.g., similarly aged subject's without Alzheimer's. In other words, for example, a subject with normal skull BMD or bone thickness exhibits a measured BMD or bone thickness that is x; the measured BMD or bone thickness from the subject being tested may be 0.5×, in which case, in some embodiments that subject's skull may be considered to have an above below level of BMD or bone thickness.

Alternatively, in some embodiments, the measured BMD or bone thickness of a subject's skull is considered below normal if an assay indicates that a particular measurement, amount, value, score, or level is about or at least about 1, 2, 3, 4, 5, 6, 7, 8, 9, 10 or more standard deviations below the measurement, amount, value, score, or level observed in skulls of subject that have normal levels BMD or bone thickness. In other cases, the measured BMD or bone thickness of a subject's skull may be considered below normal if a measurement, amount, value, score, or level of BMD or bone thickness is or is at most 2, 3, 4, 5, 6, 7, 8, 9, 10, 15, 20, 25, 30, 35, 40, 45, 50 or more times less than the measurement, amount, value, score or level of BMD or bone thickness in normal the skull of normal subjects.

In some embodiments, decreased levels of BMD or bone thickness in a subject's skull can be correlated to an advanced stage of Alzheimer's disease when compared to a control level or value. In one particular example, Alzheimer's disease can be categorized into early, middle, and late stage (also referred to as “severe” or “advanced” stage) Alzheimer's disease, any of which may be correlated to the BMD or bone thickness measured in a subject's skull. In some embodiments, a lower level of BMD or bone thickness compared to a control value may correspond to a more advanced stage of Alzheimer's disease in a subject.

It is contemplated that subject's diagnosed as having Alzheimer's disease or having an increased risk of Alzheimer's disease by measuring the BMD or bone thickness in the subject's skull can be effectively treated using PHTR1 agonist as described herein. Therefore, some embodiments a method of treating Alzheimer's disease in a subject can include the steps of: (1) measuring bone mineral density (BMD) or bone thickness of the skull of the subject; and (2) administering to the subject a therapeutically effective amount of at least one parathyroid hormone type 1 receptor (PTH1R) agonist if the measured BMD or bone thickness of the skull of the subject decreased or reduced compared to a control.

Since the increase in bone deficits found in mouse models of AD were found to be diminished upon PTHR1 agonist treatment, it is further contemplated that a reduction of one or more bone deficits, can serve as a surrogate marker of therapeutic efficacy for PTHR1 agonists described herein. It is contemplated that an agent, such as a PTHR1 agonist described herein, that can reduce one or more bone deficits in a subject is an effective Alzheimer's disease therapeutic.

Therefore, another embodiment relates to a method of determining the efficacy of an PTHR1 agonist therapy in treating Alzheimer's disease. The method includes: (1) administering an Alzheimer's disease therapeutic to a subject; (2) measuring a level of one or more of BMD or bone thickness of the skull of the subject and/or a serum bone resorption marker or a serum bone formation marker in the subject; and (3) comparing the measured level with a control sample level wherein an increased level of BMD, bone thickness and/or a serum bone resorption marker and/or the decreased level of a serum bone resorption marker is indicative of efficacy of the PTHR1 agonist therapy in treating Alzheimer's disease.

In some embodiments, the measured serum bone resorption marker can include pyridinoline (PYD). The measured serum bone formation marker can include osteocalcin. Serum bone markers may be measured from a blood sample obtained from the subject by various well-known methods.

In some embodiments, the method includes administering the PTHR1 agonist to a subject prior to measuring BMD, bone thickness a serum bone resorption marker, and/or a serum bone formation marker in the subject. Alternatively, the level or amount of BMD, bone thickness and/or serum bone markers may already be known (e.g., measured before administration of the PTHR1 agonist), and consequently those levels or amounts would be evaluated to make a determination regarding efficacy of the PTHR1 agonist. This information could then be used to select for subjects who are likely to respond to given therapeutics targeting PTH1R activity, PTH1R function, PTH1R signaling or PTH1R expression, such as the PTH1R agonists in accordance with a method described herein.

In other embodiments, the PTH1R agonist described herein can be administered in combination with an anti-Alzheimer's agent. The term “anti-Alzheimer's agent” or “anti-Alzheimer agent”, as employed herein refers to any compound that can be employed for the treatment of Alzheimer's disease and other dementias; such as, but not limited to, N-methyl-D-aspartate receptor (NMDA) receptor antagonists, acetyl cholinesterase inhibitor, acetylcholine synthesis modulators, acetylcholine storage modulators, acetylcholine release modulators, Aβ inhibitors, Aβ plaque removal agents, inhibitors of Aβ plaque formation, inhibitors of amyloid precursor protein processing enzymes, β-amyloid converting enzyme inhibitors, β-secretase inhibitors, β-secretase modulators, nerve growth factor agonists, hormone receptor blockade agents, neurotransmission modulators, and combinations thereof. In one embodiment, the anti-Alzheimer's agent is an NMDA receptor antagonist. In one embodiment, the NMDA receptor antagonist includes, but not limited to, memantine, amantadine, neramexane (1,3,3,5,5-pentamethylcyclohexan-1-amine), ketamine, rimantidine, eliprodil, ifenprodil, dizocilpine, remacemide, riluzole, aptiganel, phencyclidine, flupirtine, celfotel, felbamate, spermine, spermidine, levemopamil, and/or combinations thereof. In another embodiment, NMDA receptor antagonist employed in the present invention is an Anti-Alzheimer agent. In one embodiment, the anti-Alzheimer's agent is an inhibitor of cholinesterase. In one embodiment, the acetylcholinesterase inhibitor includes, but is not limited to, donepezil, tacrine, rivastigmine, galantamine, physostigmine, neostigmine, Huperzine A, icopezil (CP-118954, 5,7-dihydro-3-[2-[1-(phenylmethyl)-4-piperidinyl]ethyl]-6H-pyrrolo-[4,5-f-]-1,2-benzisoxazol-6-one maleate), ER-127528 (4-[(5,6-dimethoxy-2-fluoro-1-indanon)-2-yl]methyl-1-(3-fluorobenzyl) piperidine hydrochloride), zanapezil (TAK-147; 3-[1-(phenylmethyl)piperidin-4-yl]-1-(2,3,4,5-tetrahydro-1H-1-benzazepin-8-yl)-1-propane fumarate), metrifonate (T-588; -) (—R-α-[[2-(dimethylamino)ethoxy]methyl]benzo[b]thiophene-5-methano-1 hydrochloride), FK-960 (N-(4-acetyl-1-piperazinyl)-p-fluorobenzamide-hydrate), TCH-346 (N-methyl-N-2-pyropinyldibenz[b,f]oxepine-10-methanamine), SDZ-220-581 ((S)-.alpha.-amino-5-(phosphonomethyl)-[1,1′-biphenyl]-3-propionic acid), and combinations thereof.

In another embodiment, the anti-Alzheimer's agent is an Aβ inhibitor, Aβ plaque removal agents, inhibitors of Aβ plaque formation, inhibitors of amyloid precursor protein processing enzymes, β-amyloid converting enzyme inhibitors, β-secretase inhibitors, β-secretase modulators.

In another embodiment, the Aβ inhibitor includes, but is not limited to, tarenflurbil, tramiprosate, clioquinol, PBT-2 and other 8-hydroxyquinilone derivatives, Aβ plaque removal agents, inhibitors of Aβ plaque formation, inhibitors of amyloid precursor protein processing enzymes, β-amyloid converting enzyme inhibitors, β-secretase inhibitors, α-secretase modulators (LY450139; N-[N-(3,5-difluorophenacetyl)-L-alanyl)-S-phenylglycine t-butyl ester), and combinations thereof. In another embodiment, the anti-Alzheimer's agent is a nerve growth factor agonist. The nerve growth factor agonist is, but not limited to, xaliproden or brain derived neurotrophic factor or nerve growth factor.

In another embodiment, the anti-Alzheimer's agent is a hormone receptor blockade agent. The hormone receptor blockade agent is, but not limited to, leuproelide or a derivative thereof.

In another embodiment, the anti-Alzheimer's agent is a neurotransmission modulator. The neurotransmission modulator is, but not limited to, ispronicline.

In still other embodiments, the PTH1R agonist can be administered in combination with Vitamin D to a subject having or at risk of Alzheimer's disease including, for example, subjects with reduced or decreased BMD or bone thickness of the skull. Previous studies revealed some comorbidity of AD and osteoporosis not only for advanced disease, but also for the incipient conditions cognitive decline and decline of bone mineral density. Other studies have found that there is a significant correlation between concentrations of biochemical osteoporosis markers in blood plasma of subjects with mild cognitive impairment and mild AD compared to subjects with primary osteoporosis and age-matched cognitively normal controls. These results point to increased bone catabolism and concomitant remodeling/anabolism unrelated to vitamin D state in mild AD, but not in mild cognitive impairment. This corroborates previous findings of comorbidity of AD with osteoporosis in the early disease course at the level of biochemical blood markers.

Vitamin D insufficiency and deficiency was associated with all-cause dementia, AD, stroke (with and without dementia symptoms), and MRI indicators of cerebrovascular disease. Moreover, vitamin D supplementation has also been associated with protecting the length of telomeres. These findings suggest a vasculo-protective role of vitamin D and the potential efficacy of administering a PTH1R agonist in combination with vitamin D to treat AD.

The invention is further illustrated by the following example, which is not intended to limit the scope of the claims.

### Example

In this example, we used 5×FAD mice, a well-characterized AD animal model that expresses human mutant APP and presenilin genes under the control of Thy1-promoter, to test PTH₁₋₃₄'s effect on treating AD for the following reasons. First, 5×FAD mice exhibit early onset Aβ based brain pathologies and cognition deficits. Second, 5×FAD (at 5/6-MO) showed a reduced trabecular, but not cortical, bone mass, with a decrease in bone formation and an increase in bone resorption. Third, 5×FAD mouse serum samples had decreased PTH, exhibiting some features of hypoparathyroidism. Interestingly, upon intermittent treatments of PTH₁₋₃₄, 5×FAD mice exhibited diminished not only bone deficits but also brain pathologies, including Aβ accumulation and deposition, glial cell activation, and brain inflammation, as well as improved learning and memory function. The PTH₁₋₃₄ attenuation of AD brain pathology (e.g., Aβ accumulation and deposition and glial cell activation) was detectable in both female and male 5×FAD, but not wild type (WT), mice. However, PTH₁₋₃₄'s improvement in learning and memory function was only observed in 5×FAD female mice at the age of ˜5-MO. Further studies demonstrated that PTH₁₋₃₄ was able to enter the brain tissue of 5×FAD mice and concentrated around astrocytes. In primary cultured astrocytes, PTH₁₋₃₄ could induce phospho-CREB, a key signaling downstream of cAMP, suppresses expression of inflammatory cytokines or chemokines, and diminishes 5×FAD-induced astrocyte senescence. Taken together, these observations suggest that intermittent PTH₁₋₃₄ treatments may act as a senolytic-like drug, reducing systemic and brain inflammation, improving cognitive function, and implicating the potential therapeutic benefits of PTH₁₋₃₄ for not only osteoporosis but also AD.

### Materials and Methods

### Animals

5×FAD [B6SJL-Tg (APPSwFlLon, PSEN1*M146L*L286V) 6799Vas/Mmjax] transgenic mice were purchased from the Jackson Laboratory (Stock No: MMRRC_034840-JAX). 5×FAD mice express mutant human amyloid beta precursor protein (APP) and human presenilin 1 (PSEN1), with a total of five mutations including the Swedish [K670N/M671L], Florida [I716V], and London [V717I] in APP and M146L and L286V in PSEN1 under the control of Thy-1 promotor. 5×FAD mice were maintained as hemizygotes on a C57BL/6 background for experiment, and litter-mate controls (5×FAD−/−) or comparable aged C57BL/6J mice with same gender were used as WT control. The C57BL/6J mice (Stock No: 000664) were bred in our laboratory using mice purchased from The Jackson Laboratory. Tg2576 mice, which express human APP695 with the KM670/671NL mutations (APPswe) under the control of a hamster prion promoter, were purchased from Taconic (Hudson, NY, USA). Tg2576 mice were also backcrossed into C57BL/6 background, and WT (C57BL/6) control were used in parallel for each experiment. Female or male mice were subjected to experiments as indicated in the text. All mice were housed in groups of no more than 5 per cage with mice of the same genotype and gender under a 12 h light/dark cycle in a room with water and a standard rodent chow diet. All experiments with animals were approved by the Institutional Animal Care and Use Committee at Case Western Reserve University.

### Experimental Design

Parathyroid hormone (human) was purchased from TOCRIS Bioscience (Cat #3011/1, Abingdon, United Kingdom). WT or 5×FAD mice (both male and female) were treated with PTH₁₋₃₄ intermittently [once daily injection of hPTH₁₋₃₄ (50 μg/Kg) or vehicle (0.9% sodium chloride) (as a control) by subcutaneous injection, starting at 2-MO, 5 days per week] as described previously. Mice were then subjected to behavioral tests and sacrificed for examinations of their bone and brain phenotypes at the indicated ages.

### Micro-Computed Tomography (μCT)

Femurs of WT and 5×FAD female mouse with or without PTH₁₋₃₄ treatment were detached and fixed overnight in 10% formalin and stored in PBS for further characterization by microCT (μCT). The excised femurs were placed vertically in a 12-mm-diameter scanning holder and scanned using the Scanco μCT40 (Scanco Medical AG). Settings parameters are adjusted as previously described: 12 m resolution, 55 kVp, and 145 μA with an integration time of 200 ms. For the cortical analysis, the bone was scanned at the midshaft of the bone for a scan of 25 slices, and the threshold for cortical bone was set at 329. The 3D reconstruction was performed using all outlined slices and analyzed by μCT evaluation program (v.6.5-2; Scanco Medical). Data were obtained on bone volume (BV), total volume (TV), BV/TV, bone density, and cortical thickness for cortical bone. For the trabecular bone, the scan was started at the growth plate and consisted of 211 slices. One hundred slices were outlined from this point, on the inside of the cortical bone, enclosing only the trabecular bone and marrow. The threshold for trabecular bone was set at 245 and the 3D analysis performed on the 100 slices. BV, TV, BV/TV, trabecular bone number (Tb. N), trabecular bone thickness (Tb. Th), and trabecular bone space (Tb. Sp) were obtained for the trabecular bone.

### Behavior Tests

Mice (female and male) at the age of ˜5-MO were subjected to behavioral studies. The experimental procedures are described as previously. Mice were transferred to the behavior test room 2 h before any test to acclimatize to the environment. All behavioral instruments were cleaned with 70% ethanol before each trial. For Y-Maze test, mice were placed at one arm end of Y-maze and allowed to freely explore the three arms for 8 min. The total arm entries and the number of tri-ads were recorded to quantify the percentage of spontaneous alternation. For novel object recognition (NOR), which tests mouse's memory ability for a novel object, 20 min were recorded to analyze the time to explore the old object vs. the new object of each mouse. Mice were released into the chamber (60×60×20 cm, W×D×H) for 20 min to acclimatize on the first day, followed by the training and testing on the second day. Mice were free to explore two identical objects in the chamber for 20 min during the training phase. The test trials were administered after delays of 3-h post-training. During test trials, one of the objects was replaced with a new object and mice were free to explore for another 20 min, and the time spent exploring the new object and the old object was recorded. The discrimination index is quantified as the novelty object preference, which is the time spent exploring the new object minus the time exploring the old object over the total exploration time.

For Morris water maze (MWM), a circular water tank (diameter of 120 cm) fulfilled with water and a 10 cm platform were used. Non-toxic white powder paint was added to water to make the surface opaque to hide the escape platform. The maze was virtually divided into 4 quadrants, one of which contained the platform 1 cm below the water surface as an escape platform. Mice were trained for 5 days, four trials per day with 20 min intervals between trials and 60 s per trial to locate the hidden platform. Then mice were left on the platform for 15 s to help them remember the location. On the 6th day, the escape platform was removed and mice were placed in water at the point farthest from the platform and recorded for 60 s. The swimming track was recorded using a video tracking system, and the time spent in each platform quadrant and the number of platform crossings were quantified. All behavioral trials were recorded by use of an overhead camera and analyzed by Etho Vision software (Etho Vision, Noldus). Mice were assigned and data were quantified, in a double-blind method.

### ELISA Assays for Osteocalcin, PYD, Human Aβ40 and Aβ42, and PTH

Submandibular blood collection was performed in anesthetized mice. A swift lancing motion is used to puncture the vessel. Blood sample (up to 200 μl) was collected with a pipette or other collection tube, allowed to stand at room temperature for 30 min and centrifuged for 15 min at 3000 rpm. Supernatant serum was aliquot and frozen at −80° C. until use. Mouse serum levels of osteocalcin (a marker for bone formation), PYD (pyridinoline, a marker for bone resorption), and PTH were measured using mouse osteocalcin Elisa kit (QUIDEL Corporation), METRA Serum PYD EIA kit (QUIDEL Corporation, San Diego, CA, USA), and mouse PTH Elisa kit (MyBioSource), respectively, following the manufacturers' instruction.

Brain homogenized lysates from the cortex and hippocampus were obtained for human Aβ40/42 Elisa assays, as previously described with modifications. In brief, the detached tissue was homogenized in modified ice-cold TBS buffer [50 mM Tris-Cl (pH 7.6), 150 mM NaCl, 1% NP-40] with Dounce homogenizers until no visible pieces were seen. The mixture was centrifuged at 12,000 g for 20 min at 4° C., and the supernatants were collected as soluble fractions for Aβ Elisa analysis. Followed by the manufacturer's instruction, human A040 and A042 levels in the brain (200 μg total protein) homogenates were measured by an A040 human ELISA kit (Invitrogen, Cat #KHB3481) and A042 human ELISA kit (Invitrogen, Cat #KHB3441). All ODs measured after the reaction were converted to their concentrations using their corresponding standard curves.

For PTH Elisa assays, cortex and hippocampus from 6-MO WT and 5×FAD female mice were minced into small pieces and rinsed in ice-cold PBS to remove excess blood thoroughly. Tissue pieces were weighed and then homogenized in PBS [tissue weight (g): PBS volume (mL)=1:9] with a glass homogenizer on ice. The homogenates were then centrifuged for 5 min at 5000 g to get the supernatant. Mouse PTH levels were then measured by the PTH Elisa kit (MyBioSource, Cat #MBS2509255) according to instructions. The optical density (OD) was measured spectrophotometrically at a wavelength of 450 nm. The concentration of PTH were calculated by comparing the OD of the samples to the standard curve.

### Brain Tissue Processing and Immunofluorescent Staining

Coronal brain sections were obtained and subjected to immunofluorescent staining, as described previously. In brief, mice were anesthetized by 3% isoflurane and transcardially perfused with phosphate buffer (PBS, 0.01 M, pH=7.4) followed by 4% (w/v) paraformaldehyde (PFA), and the dissected brains were post-fixed with 4% PFA overnight at 4° C. Brain tissues were sectioned into 40 m-thick free-floating coronal sections for different purposes using a vibratome (Leica VT1000S). All brain slices were sequentially collected and stored at −20° C. in cryoprotectant solution (FD Section Storage Solution) for further use.

For immunostaining, free-floating sections were washed in PBS (3-5 min, 3 times) and incubated in blocking buffer (0.02% Triton X-100, and 2% donkey serum in PBS) for 1 h, then incubated in primary antibody solution overnight at 4° C. Sections were washed 3 times in PBS and incubated with corresponding conjugated secondary antibodies (1:200 in blocking buffer) for 2 h at room temperature. DAPI was used for nucleus staining. Stained sections were imaged by a confocal microscope at room temperature. Fluorescent quantification was performed using ZEN software according to the manufacturer's instructions (Carl Zeiss). The following primary antibodies were used: anti-ATG9A (ab108338, rabbit), anti-IBA1 (ab178846, rabbit), anti-IBA1 (ab5076, goat), and LPL (ab21356, mouse) from Abcam (Cambridge, MA, USA); anti-GFAP (#12389, rabbit) from Cell Signaling (Danvers, MA, USA); SLC16A1 (TA321556, rabbit) from OriGeine (Rockville, MD, USA); MAP2 (#556320, mouse) from BD Pharmingen (San Diego, CA, USA). Secondary antibodies were purchased from Jackson ImmunoResearch Laboratories (West Grove, PA, USA).

### Aβ Deposition Assay

Thioflavin S (Thio-S) staining was used for Aβ deposition assay as previously described with minimal modification. Beginning with the lateral-most section in the ROI (region of interest), every sixth tissue section of six consecutive sections was used. Free-floating brain slices were washed in PBS (3-5 min, 3 times) and incubated in 0.1% Thio-S solution (dissolved in ddH2O and filtered) for 10 min at room temperature in the dark. The brain slices were then washed with a series of graded EtOH in the dark as follows: 95% EtOH for 2 min, 80% EtOH for 2 min, and 70% EtOH for 2 min. Finally, the brain slices were washed with PBS 3 times in the dark and transferred to slides for sealing with an antifade fluorescence mounting medium. All the brain slices were captured on a Laser confocal microscopy. The images were adjusted to the same threshold to increase signal-to-noise ratio. Plaques >5.5 μm² were quantified. Four sections per mouse were quantified. The layers or sub-regions of cortex and hippocampus in each image were outlined and analyzed with the National Institutes of Health ImageJ software.

### Western Blot Analysis

Total proteins from cortex, hippocampus, or cultured astrocytes were extracted using RIPA lysis buffer [50 mM Tris-HCl (pH7.5), 150 mM NaCl, 1 mM EDTA, 1% v/v NP-40, 0.1% SDS, 1% sodium deoxycholate supplemented with Protease Inhibitor Cocktail (Roche) and 1 mM PMSF] and analyzed by Western Blot. Lysates were cleared by centrifugation at 12,000 g for 15 min at 4° C. to remove debris and to obtain homogenates. Homogenates samples were resolved by SDS-PAGE (8˜15%) gels and then electroblotted onto nitrocellulose membranes (Cat #1620112, Bio-Rad Laboratories). Membranes were blocked with 5% BSA (w/v) in TBS-Tween (50 mM Tris, 150 mM NaCl, pH7.6 and 0.1% v/v Tween-20) for 1 h and then probed with indicated primary antibodies overnight in 4° C. Membranes were washed with TBST 4 times and incubated with HRP-conjugated secondary antibodies (1:5000 in TBST) at room temperature for 1 h. After washing with TBST 3 times, antibody reactivity was detected by the Enhanced Chemiluminescence (ECL) detection system (Amersham Biosciences). Band density of proteins was normalized in relation to loading control and analyzed using NIH ImageJ software. Primary anti-bodies used were as follows: IBA1 (ab178846, rabbit), p16 (ab51243, rabbit), p53 (ab131442, rabbit), and anti-beta Actin (ab8226, mouse) from Abcam (Cambridge, MA, USA); GFAP (12389, rabbit), p-CREB (Ser133) (9198S, rabbit), p-AKT(S473) (4060P, rabbit), AKT (9272S, rabbit) and GAPDH (97166S, mouse) from Cell Signaling (Danvers, MA, USA); PTH1R (PA5-77,689, rabbit) from Invitrogen (Carlsbad, CA, USA); p-ERK1/2 (sc-136521, mouse) and ERK1/2 (sc-514302, mouse) from Santa Cruz Biotechnology (Santa Cruz, CA, USA). Secondary anti-bodies were purchased from Jackson ImmunoResearch Laboratories (West Grove, PA, USA).

Reverse Transcription-Quantitative Polymerase Chain Reaction (qRT-PCR)

Total RNA was extracted from cortex, hippocampus, or cultured astrocytes using a TRizol reagent (Invitrogen, cat #15596-026, Carlsbad, CA, USA) as previously described. Purified RNA (1-5 μg) was used for cDNA synthesis with the Revert Aid First Strand cDNA Synthesis Kit (Thermo Scientific, cat #K1621, Waltham, MA, USA). The cDNA products were subjected to subsequent quantitative PCR (qPCR) using the QuantiFast SYBR Green PCR Kit (QIA-GEN, cat #204057, Hilden, Germany) with a qPCR System (StepOne Plus). Each sample was repeated at least 3 times, and the mRNA level was normalized to GAPDH using the 2^{-ΔΔCt} method.

### Mouse Serum Cytokine Array

Serum samples from 5×FAD, Tg2576, and their WT control female mice (6˜MO) were collected and used for serum cytokine detection. Cytokines were measured with Mouse Cytokine Array Panel A (R&D Systems, cat #ARY006, Minneapolis, MN, USA). Briefly, the mixture of serum and the biotinylated detection antibodies cocktail is incubated with a Mouse Cytokine Array membrane. Any cytokine/detection antibody complex present was bound on the membrane by its cognate immobilized capture antibody. After washing off the unbound protein, streptavidin-horseradish peroxidase and chemiluminescent detection reagents were added to generate light signals. The light produced at each spot is proportional to the amount of cytokine binding.

### Biotin-PTH1-34 Diffusion Experiment

Biotin-labeled PTH₁₋₃₄ (ANASPEC, cat #AS-20690, Fremont, CA, USA) was used to detect PTH diffusion and function. 2.5˜MO old WT and 5×FAD female mice were anesthetized and given a total of 100 μg of Biotin-PTH₁₋₃₄ or vehicle (Phosphate Buffer) by tail-intravenous injection. After 30 min, mice were perfused with 4% PFA (pH 7.4), and the brain slices were obtained as described above. Immunofluorescent staining was used to label the biotin signal using a biotin antibody. All the brain slices were captured on laser confocal microscopy.

### Astrocyte Culture and In Vitro PTH₁₋₃₄ Treatment

Astrocyte culture was performed according to the standard protocol with some modifications as described in previous literature. In brief, the cerebral cortex and hippocampus of postnatal day 3 (P3) female pups were dissected under a stereomicroscope and placed in ice-cold calcium- and magnesium-free HBSS (Hanks Balanced Salt Solution). The tissues were digested with 2.5% trypsin-EDTA, 3 ml for each mouse, at 37° C. for 20 min with shaking slightly every 5 min. The digested tissue was blown into single-cell suspension, filtered through a 70-mm filter mesh, pelleted at 900×g for 5 min, and resuspended in growth medium (DMEM plus 10% FBS), which was incubated at 37° C. with 5% CO₂. Three cortical and hippocampal tissues per 100 mm culture dish were necessary to achieve a proper astrocyte density. The culture medium was changed after 24 h and every 3 days following the initial change to remove non-astrocyte cells. Confluent cells were passed after 7 days using 0.05% trypsin-EDTA and could be used for experiments after another 3-6 days of culture. Cultured astrocytes were treated with 100 ng/ml of PTH₁₋₃₄, and samples were collected at indicated times to detect changes in proteins, genes, and cells.

### RNA Scope

The RNA scope was performed in mouse brain using the RNAscope® Multiplex Fluorescent Detection Kit (Cat #PN323110, Noble Park North, Australia). The probes and hybrid oven were purchased from the ACDbio company, and all the procedures were performed according to the manufacturer's protocol and as described previously. Briefly, 6˜MO WT and 5×FAD female mice were deeply anesthetized and perfused with 4% PFA. The dissected brains were post-fixed with 4% PFA overnight and dehydrated with 30% sucrose in PBS. Then, the brains were cryo-sectioned into 12 m sections using a freezing microtome (Leica, Buffalo Grove, IL, USA). Every sixth tissue section of six consecutive sections in ROI was used for staining and followed the RNAscope® Multiplex Fluorescent Reagent Kit v2 User Manual processing. Finally, sections were imaged with Zeiss LSM 800 system.

### Senescence Associated β-Gal Staining

SA-β-gal staining of cultured astrocytes was performed as previously reported. SA-β-gal staining was performed using an SA-β-gal staining kit (Cell Signaling, cat #9860, Danvers, MA, USA) according to the manufacturer's instructions. All stained cell slides were captured on a BZX slider scanner. The SA-β-gal intensity was quantified by the NIH ImageJ software.

### Statistical Analyses

All data were presented as mean±SD. For in vivo studies, three to eight mice per group per assay were used, and the gender of the mice was indicated in the experiment. For behavior tests, 6-8 mice per group were used, and the tests were conducted separately for different gender. For in vitro cellular biological and biochemical studies, each experiment was repeated at least three times. The numbers of biological replicates are described in the figure legends. All immunofluorescence staining and immunoblotting data were quantified by the NIH ImageJ software. Statistical analyses were performed using Prism 7.0 (GraphPad Software). For two independent data comparisons, unpaired Student's t-test was used to determine statistical significance. For multiple comparisons of three or more sets of samples, one-way ANOVA or two-way ANOVA test were used. Differences were considered significant when P<0.05 (*P<0.05, **P<0.01, ***P<0.001).

### Results

Trabecular Bone Loss in 5×FAD Mice, which was Attenuated by PTH₁₋₃₄ Treatments

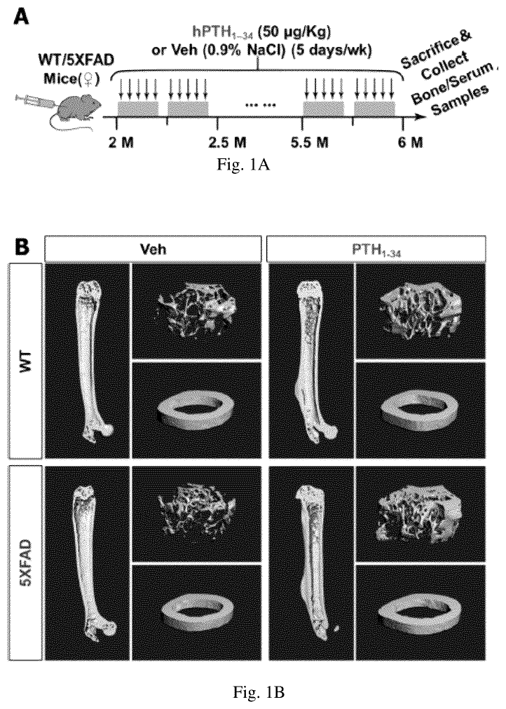

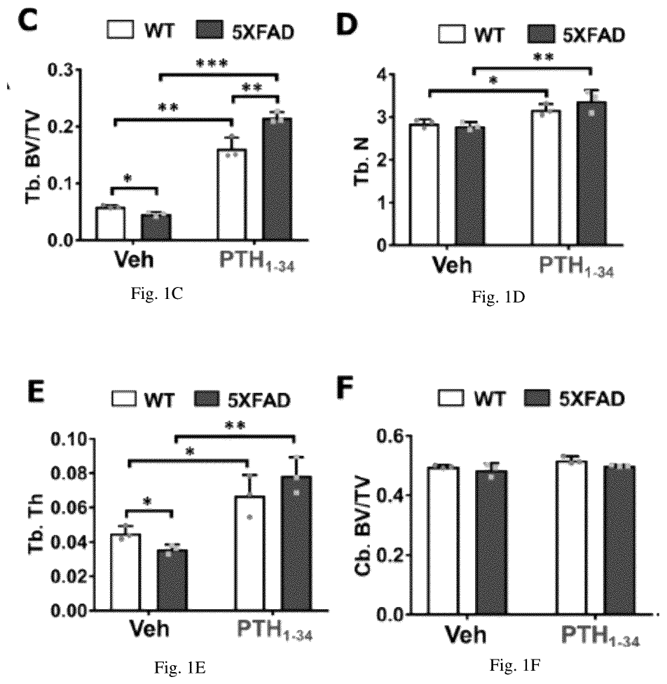

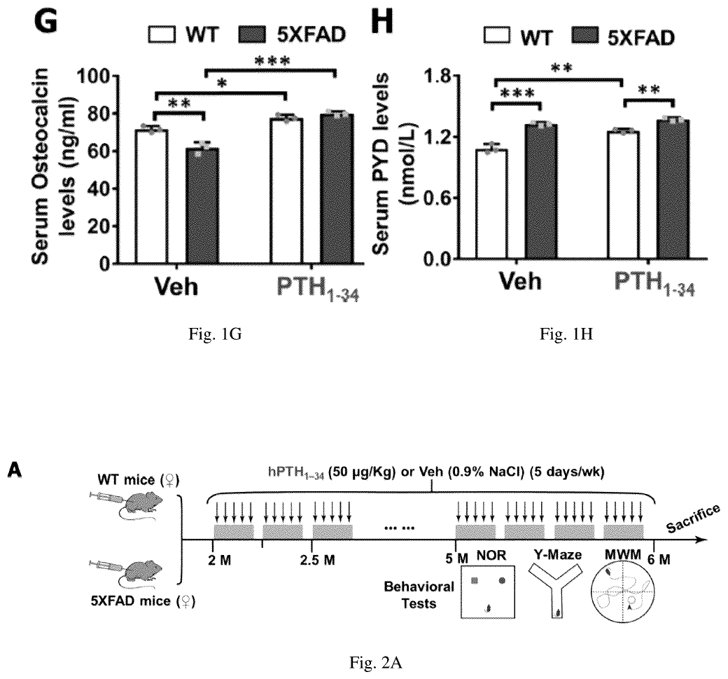

To further address whether AD-relevant mouse models show any skeletal bone deficit and to investigate whether PTH₁₋₃₄ treatments affect AD-associated bone deficits, we used 5×FAD mouse, a well-characterized AD animal model that expresses mutant human APP and presenilin genes under the control of Thy1-promoter and exhibit early onset Aβ based brain pathologies (˜2 MO) and cognition deficits (˜4 MO). C57BL/6J mice were used as WT controls. We first examined bone phenotypes by microCT (CT) analyses in femurs of control/WT and 5×FAD female mice, in light of reports of earlier onset and severer phenotypes in female than male 5×FAD mice. The control/WT and 5×FAD mice were treated with Vehicle (0.9% NaCl) or PTH₁₋₃₄ intermittently (once per day and five days per week), starting at age of 2-MO and sacrificed at ˜6-MO, as illustrated in FIG. 1A. Notice that in the absence of PTH₁₋₃₄, CT examinations showed reductions in trabecular bone volumes (Tb. BV/TV) and trabecular bone thickness (Tb. Th), without changes in their trabecular bone numbers (Tb. N) and cortical bone volumes (Cb. BV/TV), in 5×FAD mice (at age of 6-MO), as compared with those of litter mate control/WT mice (FIG. 1B-F), suggesting a bone deficit in 5×FAD mice. In line with this view were observations that serum levels of osteocalcin (a marker for bone formation) were decreased, but PYD (pyridinoline) (a maker for bone resorption) levels were increased, in 5×FAD mice (FIG. 1G-H). These results thus implicate both reduced bone formation and elevated bone resorption to underlie the trabecular bone-loss in 5×FAD mice.

Interestingly, upon PTH₁₋₃₄ treatments, more trabecular bone mass was induced in 5×FAD mice than that in WT mice (FIG. 1B-C), suggesting an enhanced PTH₁₋₃₄ response in 5×FAD mice. In line with this view were the observations that PTH₁₋₃₄ increased serum osteocalcin levels more in 5×FAD mice than that in control mice (FIG. 1G), but had little to no effect on serum PYD levels in 5×FAD mice, as compared to that of WT controls (FIG. 1H). These results thus suggest that the PTH₁₋₃₄-induced trabecular bone mass in 5×FAD mice is likely due in large part to the elevated bone formation. The two-way ANOVA analysis showed that PTH₁₋₃₄ treatment has a significant effect on various measures of bone phenotypes (P<0.05) and that genotype has a significant effect on Tb. B/TV, serum osteocalcin levels, and serum PYD levels (P<0.05) (Table 1). Significant interactions between PTH₁₋₃₄ treatment×genotype have been observed in Tb. BV/TV (F=27.03, p=0.0008), serum osteocalcin levels (F=26.59, p=0.0009), and serum PYD levels (F=17.01, p=0.0033) (Table 1). These results suggest an enhanced bone metabolism in response to PTH₁₋₃₄ in 5×FAD mice.

TABLE 1
Two-Way ANOVA analysis of bone phenotype

| Analysis | | | | | |
|---|---|---|---|---|---|
| Item | Source | DF | MS | F | P Value |
| Tb. BV/TV | PTH₁₋₃₄ treatment | 1 | 0.05523 | 437.8 | <0.0001 |
| | Genotype | 1 | 0.001296 | 10.27 | 0.0125 |
| | Interaction | 1 | 0.00341 | 27.03 | 0.0008 |
| Tb. N | PTH₁₋₃₄ treatment | 1 | 0.6101 | 24.63 | 0.0011 |
| | Genotype | 1 | 0.01696 | 0.6847 | 0.4320 |
| | Interaction | 1 | 0.05512 | 2.226 | 0.1741 |
| Tb. Th | PTH₁₋₃₄ treatment | 1 | 0.003104 | 43.89 | 0.0002 |
| | Genotype | 1 | 4.32e−006 | 0.06108 | 0.8110 |
| | Interaction | 1 | 0.0003308 | 4.677 | 0.0625 |
| Cb. BV/TV | PTH₁₋₃₄ treatment | 1 | 0.0009541 | 5.536 | 0.0465 |
| | Genotype | 1 | 0.0006308 | 3.66 | 0.0921 |
| | Interaction | 1 | 3.333e−005 | 0.1934 | 0.6717 |
| Serum | PTH₁₋₃₄ treatment | 1 | 441.9 | 106.4 | <0.0001 |
| Osteocalcin | Genotype | 1 | 42.26 | 10.18 | 0.0128 |
| | Interaction | 1 | 110.4 | 26.59 | 0.0009 |
| Serum | PTH₁₋₃₄ treatment | 1 | 0.03663 | 48.91 | 0.0001 |
| PYD | Genotype | 1 | 0.09487 | 126.7 | <0.0001 |
| | Interaction | 1 | 0.01274 | 17.01 | 0.0033 |

Source: Source of Variation,
DF: Degrees of Freedom,
MS: Mean Square,
F: F-statistic,
*P < 0.05 significance.

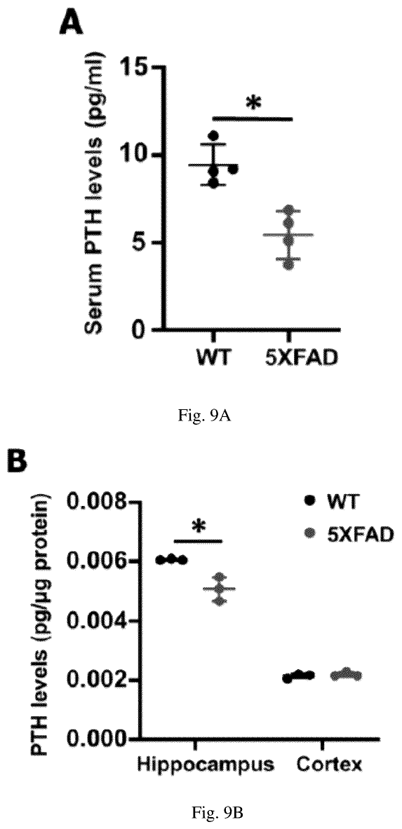

To understand how PTH₁₋₃₄ induced anabolic response is enhanced in 5×FAD mice, we measured mouse PTH levels in control and 5×FAD female mouse serum samples and brain (cortex and hippocampus) homogenates (at age of 6˜MO). Interestingly, ELISA showed lower levels of PTH in 5×FAD serum and hippocampal samples than those of controls but comparable levels of PTH in 5×FAD cortex to that of controls (FIGS. 9A-B). These results suggest that PTH is detectable in the brain, but it is reduced in 5×FAD serum and hippocampus. Such a PTH-deficiency may underlie the enhanced response to the injected hPTH₁₋₃₄.

### PTH₁₋₃₄ Attenuation of Cognition Decline and Memory-Loss in 5×FAD Mice

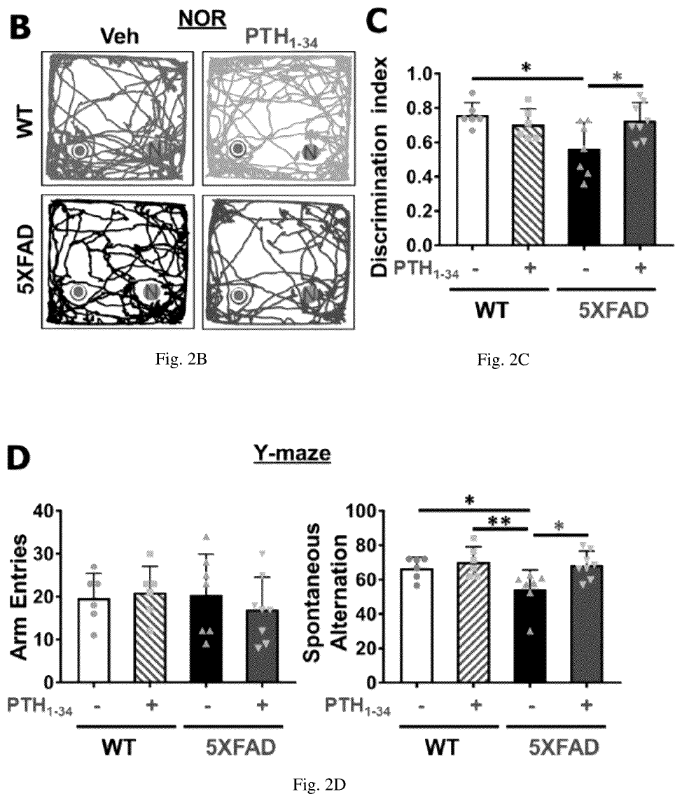

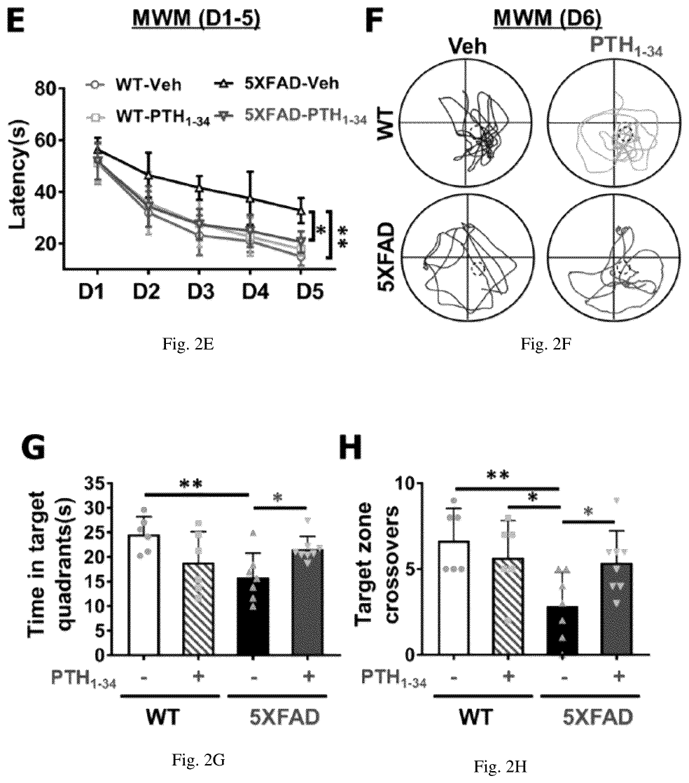

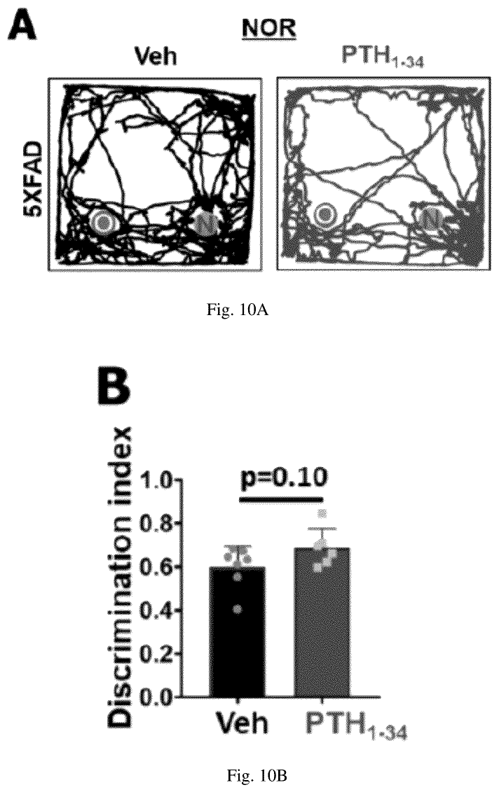

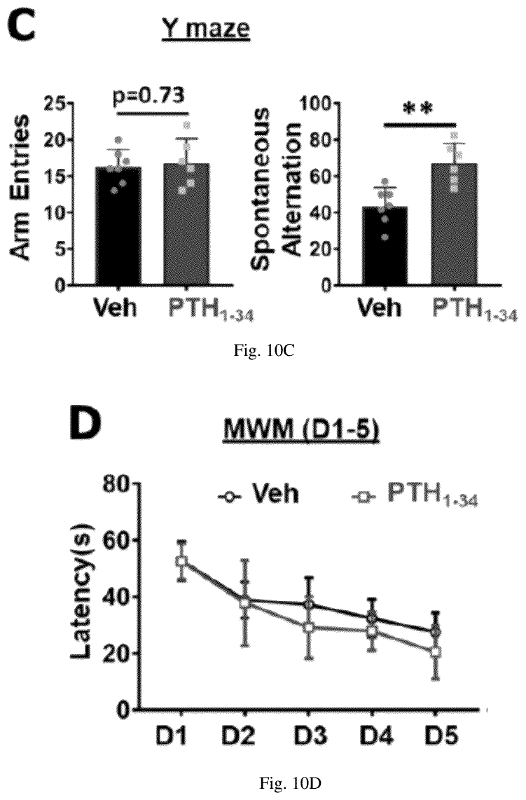

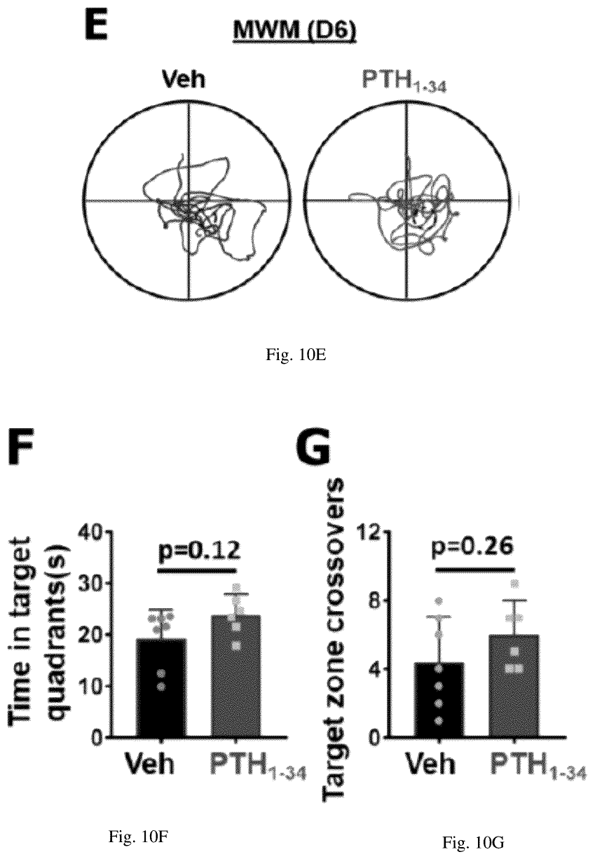

AD is a progressive neurodegenerative disease commonly associated with memory deficits and cognitive decline. We thus asked whether PTH₁₋₃₄ treatments could improve cognitive function in 5×FAD mice. WT or 5×FAD female mice were administered with hPTH₁ 34 or Veh intermittently as illustrated in FIG. 2A, and behavioral experiments began when the mice were ˜5-MO. Novel object recognition (NOR) and Morris water maze (MWM) tests were used to access mouse cognitive function or learning and memory, and Y-maze was used to test mouse working memory. While the cognitive function was substantially reduced in 5×FAD female mice compared to WT mice of the same age, these declines were largely brought down by PTH₁₋₃₄ (FIG. 2B-H). Upon treatments with PTH₁₋₃₄, 5×FAD female mice performed significantly better during these tests, as compared with those of 5×FAD control mice, exhibiting improved recognition of the novel object (FIG. 2B-C), faster learning of the hidden platform during MWM tests (FIG. 2E), and better spatial memory of the platform location (FIG. 2F-H). Additionally, these PTH₁₋₃₄ treated 5×FAD female mice showed an increase in spontaneous alternation, but comparable arm entries to those of Veh-treated 5×FAD female mice, during Y-maze tests (FIG. 2D), suggesting an improvement in spatial working memory. The two-way ANOVA analysis showed the effect of genotype (5×FAD) in the Y maze (spontaneous alternation: F=4.283, P=0.0499) and MWM test (target zone crossovers: F=7.363, P=0.0124; time in target quadrant: F=3.095, P=0.0918), while the significant effect of PTH₁₋₃₄ treatment only detected in the Y maze (spontaneous alternation: F=6.832, P=0.0155). There were interactive effects between PTH1-34 treatment×genotype on NOR (discrimination index: F=6.379, P=0.0189) and MWM test (target zone crossovers: F=5.417, P=0.0291; time in target quadrant: F=11.15, P=0.0028) (Table 2). The PTH₁₋₃₄ improvement of the cognitive function in 5×FAD mice appeared to be more pronounced in females than males (FIG. 2 and FIG. 10). Little to no improvement in cognitive function in novel object recognition (FIG. 10A-B) nor spatial learning and memory in MWM (FIG. 10D-G) was detected by PTH₁₋₃₄ treatments in 5×FAD male mice, although PTH₁₋₃₄ had a positive effect on the spontaneous alternation during Y maze tests (FIG. 10C). In aggregates, these intriguing results suggest that PTH₁₋₃₄ improves cognitive function in 5×FAD mice, which is currently manifested primarily in female mice.

TABLE 2
Two-Way ANOVA analysis of behavior tests of female mice

| Analysis | | | | | |
|---|---|---|---|---|---|
| Item | Source | DF | MS | F | P Value |
| NOR | PTH₁₋₃₄ | 1 | 0.02056 | 1.646 | 0.2122 |
| (Discrim- | treatment | | | | |
| ination | Genotype | 1 | 0.05092 | 4.078 | 0.0553 |
| Index) | Interaction | 1 | 0.07966 | 6.379 | 0.0189 |
| Y maze | PTH₁₋₃₄ | 1 | 7.302 | 0.1293 | 0.7225 |
| (Total Arm | treatment | | | | |
| Entries) | Genotype | 1 | 17.44 | 0.3088 | 0.5838 |
| | Interaction | 1 | 37.72 | 0.6678 | 0.4222 |
| Y maze | PTH₁₋₃₄ | 1 | 531.5 | 6.832 | 0.0155 |
| (Spontaneous | treatment | | | | |
| Alternation) | Genotype | 1 | 333.2 | 4.283 | 0.0499 |
| | Interaction | 1 | 187.4 | 2.409 | 0.1343 |
| MWM | Training | 4 | 3882 | 109 | <0.0001 |
| (Latencies) | Time | | | | |
| | Groups | 3 | 1397 | 12.92 | <0.0001 |
| | Interaction | 12 | 38.89 | 1.092 | 0.3765 |
| MWM | PTH₁₋₃₄ | 1 | 0.00186 | 9.489e−005 | 0.9923 |
| (Time in | treatment | | | | |
| Target | Genotype | 1 | 60.68 | 3.095 | 0.0918 |
| Quadrant) | Interaction | 1 | 218.6 | 11.15 | 0.0028 |
| MWM | PTH₁₋₃₄ | 1 | 3.832 | 1.008 | 0.3257 |
| (Target Zone | treatment | | | | |
| Crossovers) | Genotype | 1 | 27.98 | 7.363 | 0.0124 |
| | Interaction | 1 | 20.58 | 5.417 | 0.0291 |

Source: Source of Variation,
DF: Degrees of Freedom,
MS: Mean Square,
F: F-statistic,
*P < 0.05 significance.

### PTH₁₋₃₄ Reduction of Soluble Aβ Level and In-Soluble Aβ Deposition in 5×FAD Brain

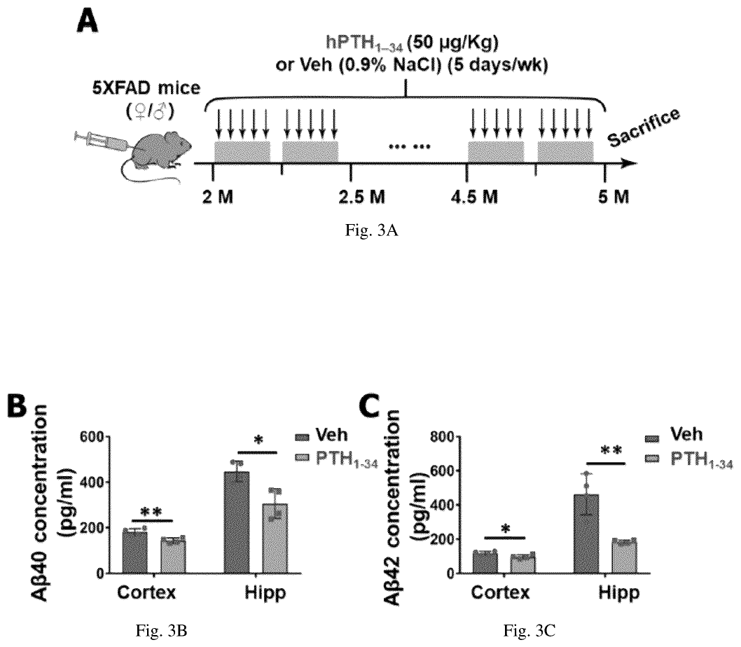

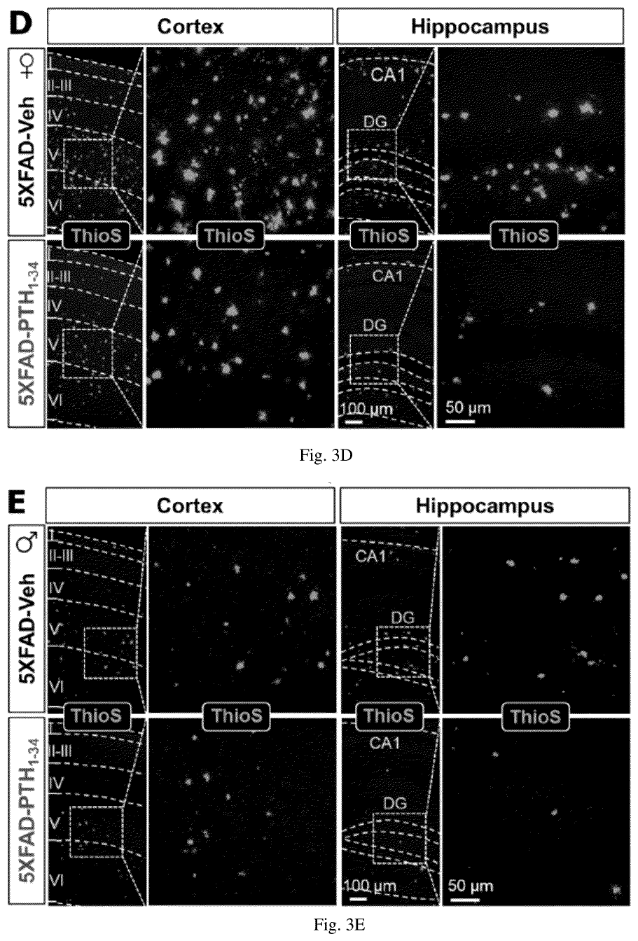

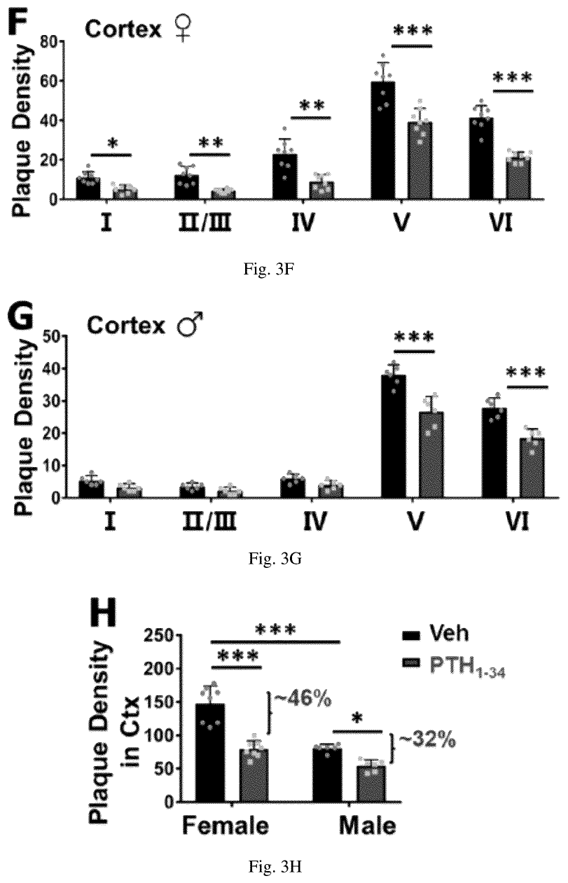

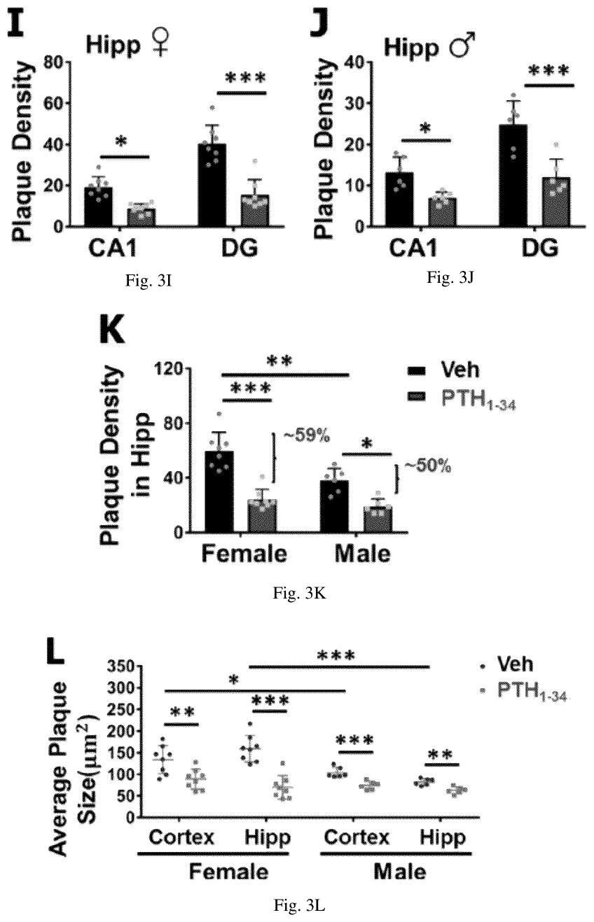

It is known that the β-amyloid accumulation and deposition in 5×FAD mice is a crucial pathology for AD development. To investigate the effect of PTH₁₋₃₄ treatments on AD-relevant brain pathology, 5×FAD mice (both male and female) were administered with hPTH1-34 or vehicle (Veh) as illustrated in FIG. 3A. We first measured both human Aβ40 and Aβ42 levels in soluble fraction from brain homogenates (cortex and hippocampus) of 5×FAD female mice in response to PTH₁₋₃₄ treatments. ELISA analyses showed significant reductions in human Aβ40 and Aβ42 levels in homogenates of both cortex and hippocampus from 5×FAD mice treated with PTH₁₋₃₄, as compared with those of Veh treatments (FIG. 3B-C), suggesting PTH₁₋₃₄'s inhibitory effect on Aβ accumulation. To determine whether the insoluble human Aβ and mouse Aβ levels are altered in 5×FAD mice by PTH₁₋₃₄ treatments, we then evaluated Aβ plaques or deposition in both groups of brain sections by Thio-S staining. In line with the view of PTH₁₋₃₄ inhibition of Aβ accumulation, PTH₁₋₃₄ treatments decreased Thio-S positive (+) Aβ plaques in cerebral cortex and hippocampus of 5×FAD female mice; both plaque density in each layer of cortex or sub-regions of hippocampus and plaque size showed significant reductions, as compared with those of Veh-treated 5×FAD female mice (FIG. 3D, F, I, L). Notice that this PTH₁₋₃₄ effect was detected in not only female but also in male 5×FAD mice (FIG. 3D-E). Plaque density (mainly in the fifth and sixth layers of the cortex and in the DG region of the hippocampus) (FIG. 3G, J) and plaque size (FIG. 3L) were all decreased in PTH₁₋₃₄-treated 5×FAD male mice, as compared with Veh-treated male mice, indicating PTH₁₋₃₄'s gender independence in this pathology. Remarkably, in the absence of the PTH₁₋₃₄, the levels of amyloid deposition (both plaque density and plaque size) in the cortex and hippocampus of female 5×FAD mice were significantly higher than those in male 5×FAD mice (FIG. 3H, K, L). Interestingly, PTH₁₋₃₄ reduction of the levels of amyloid deposition appeared to be more dramatically in female than those of male 5×FAD mice, with ˜46% and ˜59% reductions in plaque density in female 5×FAD cortex and hippocampus, respectively, while ˜32% and ˜50% reductions in male 5×FAD cortex and hippocampus, respectively, and ˜34% and ˜56% reductions in plaque size in female 5×FAD cortex and hippocampus, respectively, while ˜28% and ˜23% reductions in male 5×FAD cortex and hippocampus respectively (FIG. 3H, K, L). Significant effects of gender and PTH₁₋₃₄ treatment were showed in both plaque density and plaque size (P<0.05), no matter in the cortex or hippocampus (Table 3). Significant interactions between PTH₁₋₃₄ treatment×gender were observed in both plaque density (cortex: F=11.17, P=0.0027; hippocampus: F=5.021, P=0.0346) and plaque size (only hippocampus: F=16.2, P=0.0005) (Table 3), indicating a sex difference in response to PTH₁₋₃₄ treatment. Together, these results suggest that intermittently PTH₁₋₃₄ treatments attenuate Aβ pathology in 5×FAD brain, and this effect is more obvious in female mice.

TABLE 3
Two-Way ANOVA analysis of Aβ deposition

| Analysis Item | Source | DF | MS | F | P Value |
|---|---|---|---|---|---|
| Plaque Density | Gender | 1 | 14457 | 53.21 | <0.0001 |
| (Ctx) | PTH₁₋₃₄ treatment | 1 | 15282 | 56.25 | <0.0001 |
| | Interaction | 1 | 3036 | 11.17 | 0.0027 |
| Plaque Density | Gender | 1 | 1250 | 13.44 | 0.0012 |
| (Hipp) | PTH₁₋₃₄ treatment | 1 | 5092 | 54.78 | <0.0001 |
| | Interaction | 1 | 466.7 | 5.021 | 0.0346 |
| Plaque Size | Gender | 1 | 3403 | 6.524 | 0.0174 |
| (Ctx) | PTH₁₋₃₄ treatment | 1 | 9742 | 18.68 | 0.0002 |
| | Interaction | 1 | 416.5 | 0.7986 | 0.3804 |
| Plaque Size | Gender | 1 | 12280 | 23.46 | <0.0001 |
| (Hipp) | PTH1-34 treatment | 1 | 20432 | 39.04 | <0.0001 |
| | Interaction | 1 | 8481 | 16.2 | 0.0005 |

Source: Source of Variation,
DF: Degrees of Freedom,
MS: Mean Square,
F: F-statistic,
*P < 0.05 significance.

### PTH₁₋₃₄ Reduction of Plaque-Associated Dystrophic Neurites, but not Plaque-Associated Microglial Cells in 5×FAD Mice

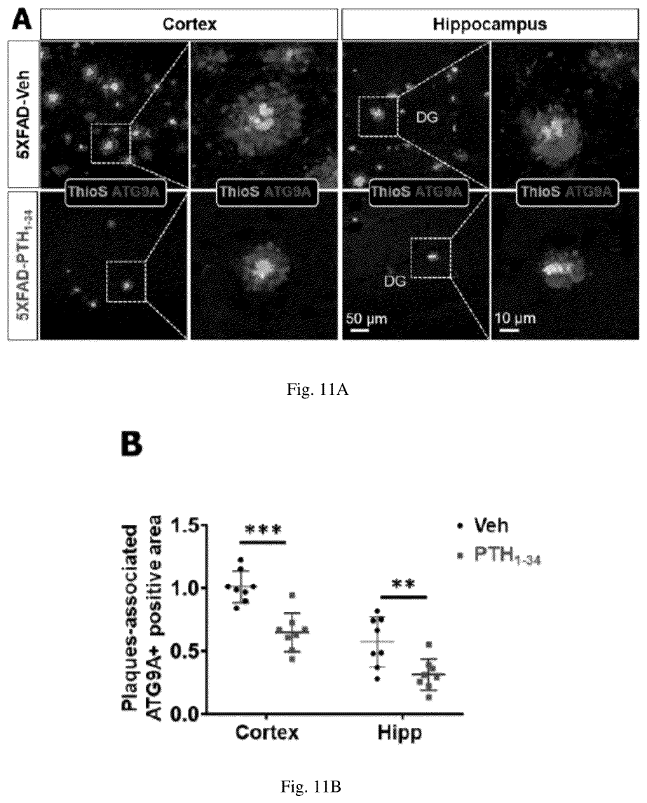

To understand how PTH₁₋₃₄ suppresses Aβ accumulation and deposition in the 5×FAD brain, we further examined Aβ plaque-associated pathology in 5×FAD female mice (˜6 MO) treated with PTH₁₋₃₄ or vehicle. Aβ plaques are surrounded by dystrophic neurites, a feature of neurodegenerative pathology, and activated glial cells, including IBA1+ microglia and GFAP+ astrocytes. Co-immunostaining analysis of ThioS with ATG9A, a pre-autophagosome protein that accumulates in the dystrophic neurites and is commonly used as a marker for dystrophic neurites, showed smaller and less dense ATG9A+ dystrophic neurites in PTH₁₋₃₄ treated 5×FAD brain (both cortex and hippocampus) than those of Veh-treated 5×FAD mice (FIG. 11A-B), indicating a reduction of dystrophic neurite formation by PTH₁₋₃₄, in line with the view of PTH₁₋₃₄ decrease of Aβ plaques.

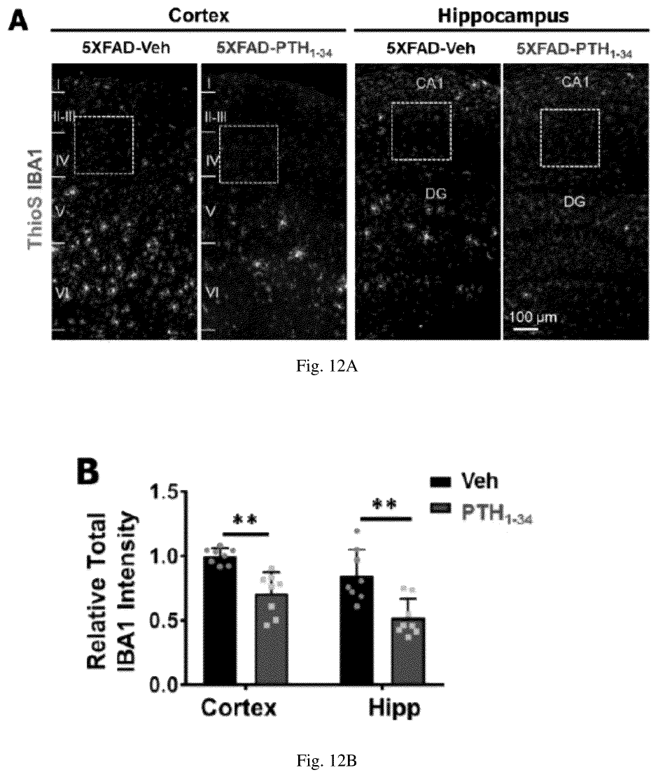

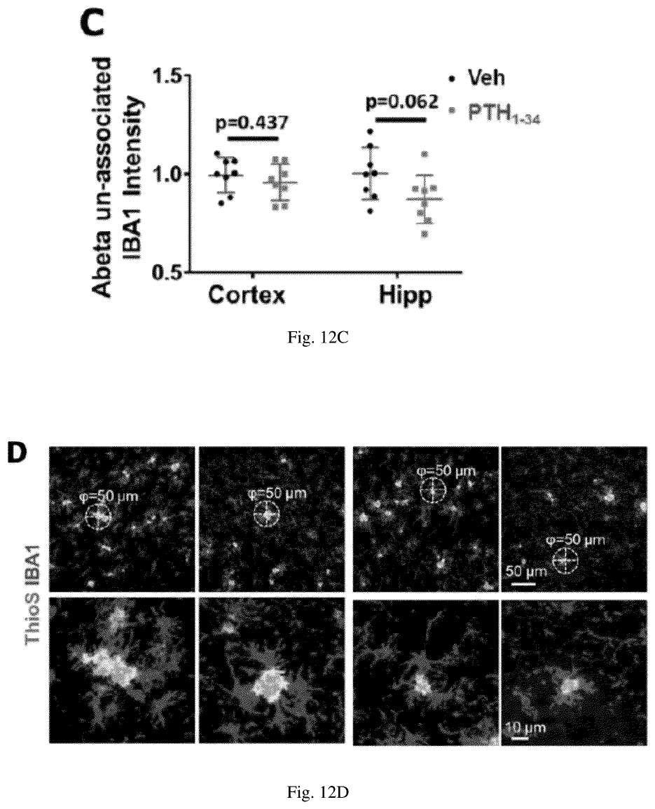

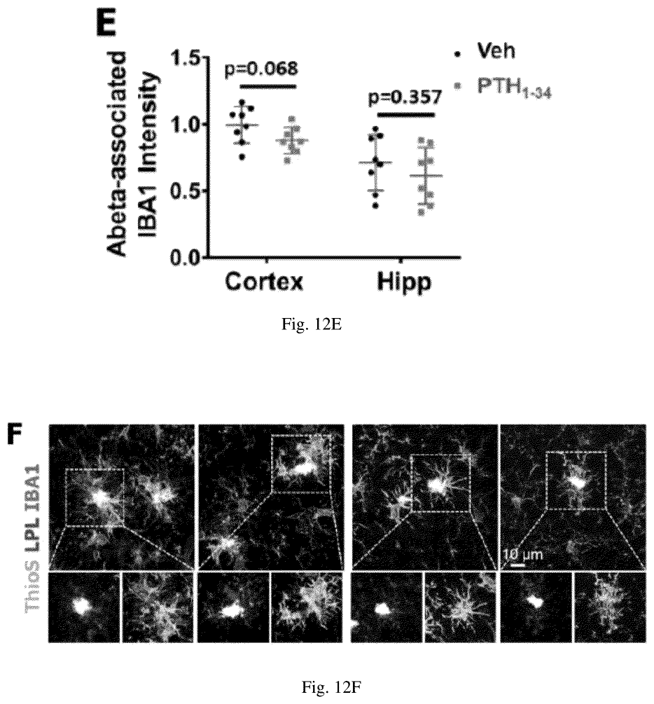

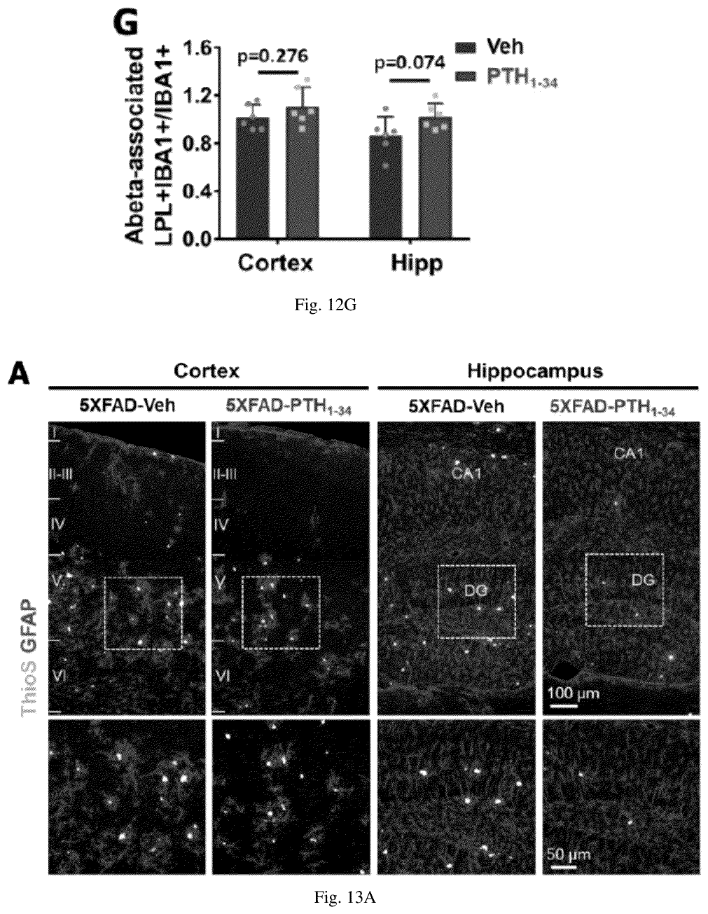

We then performed a co-immunostaining analysis of ThioS with IBA1, a marker for microglial cells. Although the overall IBA1 fluorescence intensity was lower in PTH₁₋₃₄ treated 5×FAD cortex and hippocampus than that of Veh controls (FIG. 12A-B), the ThioS+Aβ plaque associated IBA1 fluorescence was unchanged by PTH₁₋₃₄ treatments (FIG. 12D-E). Additionally, the IBA1+cell density and fluorescence intensity in regions without Aβ plaques were also comparable in PTH₁₋₃₄ treated 5×FAD mice to those of Veh treatments (FIG. 12C). These results suggest that the overall reduction in IBA1 fluorescence intensity may be due to the reduced number of Aβ plaques.

Notice that the Aβ plaque-associated microglial cells are also called DAM (Degeneration Associated Microglia), because of their unique distribution pattern and expression of molecular features (e.g., higher expressions of genes such as LPL, but lower levels of TMEM119 and CX3CR1, than those of Aβ-un-associated microglia or resting microglia). As DAMs are implicated in promoting Aβ clearance, we further examined whether DAMs are affected by PTH₁₋₃₄ treatments. Co-immunostaining analysis of IBA1 and LPL (a marker for DAM) with ThioS showed little to no difference of Aβ associated LPL+IBA1+ microglial cells in the brain between PTH₁₋₃₄ and Veh-treated 5×FAD mice (FIG. 12F-G), suggesting a little effect on DAM formation by PTH₁₋₃₄. These results demonstrate decreased amyloid plaques accompanied by decreased neural toxicity in PTH₁₋₃₄ treated 5×FAD mice, suggesting PTH₁₋₃₄'s function in preventing both Aβ accumulation and dystrophic neurites formation.

PTH₁₋₃₄ Diminishment of GFAP⁺ Reactive Astrocytes and Brain Inflammation in 5×FAD Mice

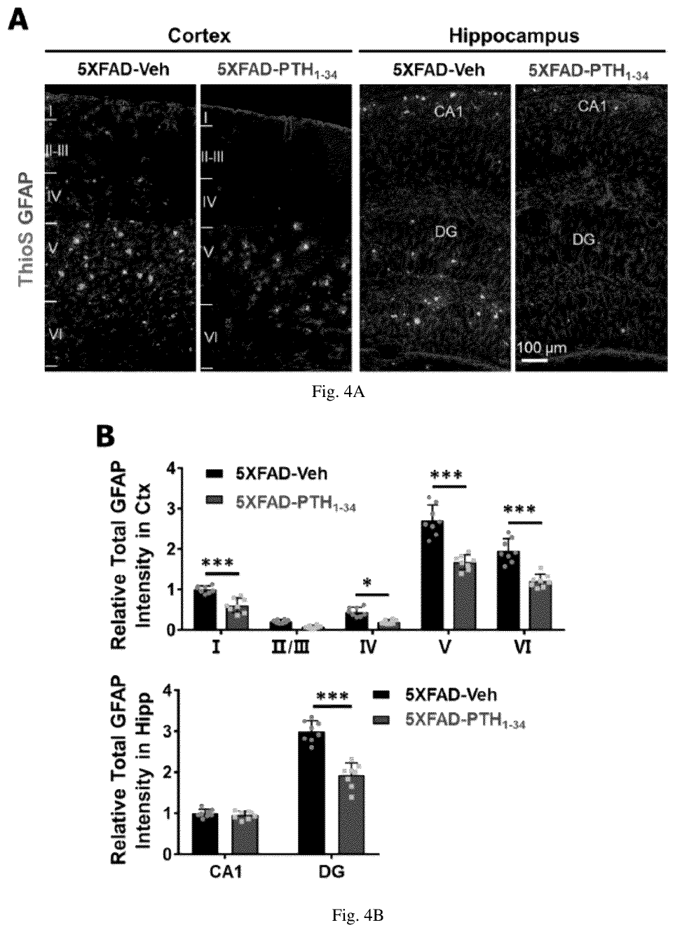

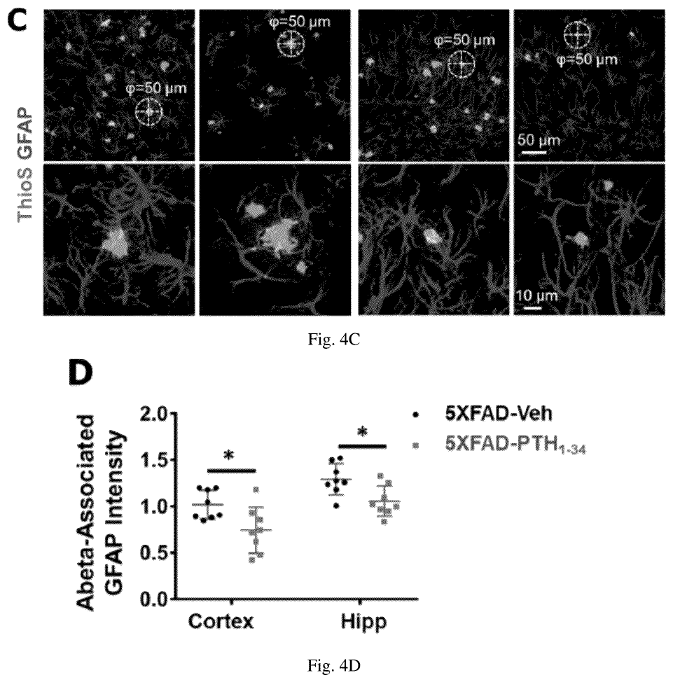

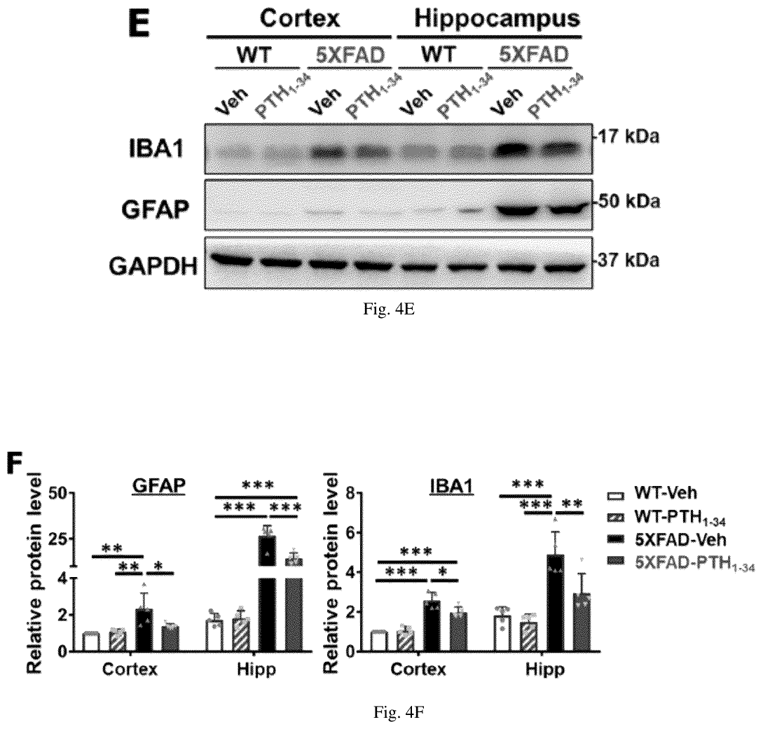

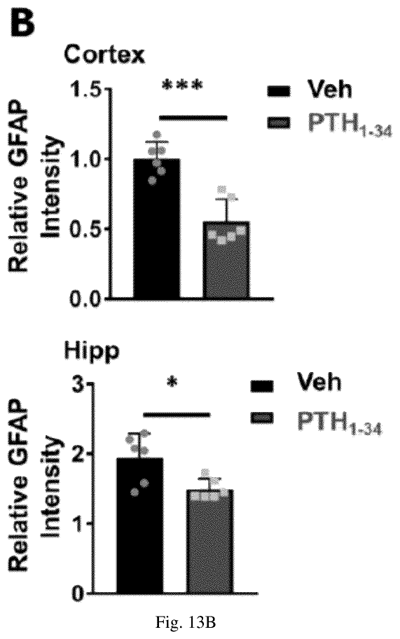

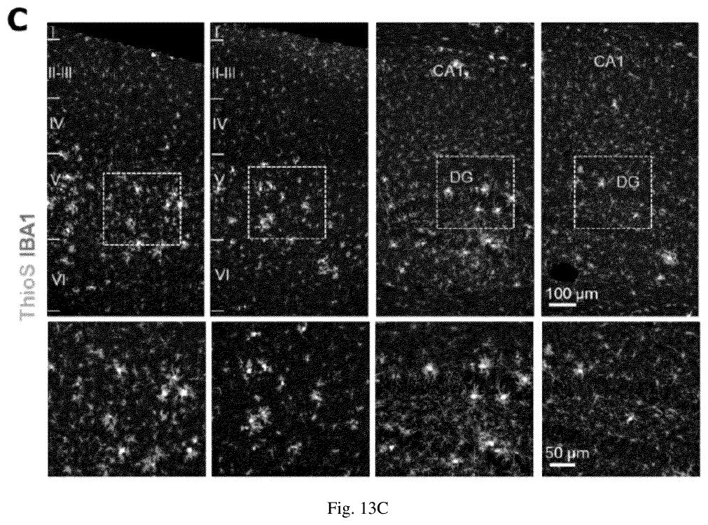

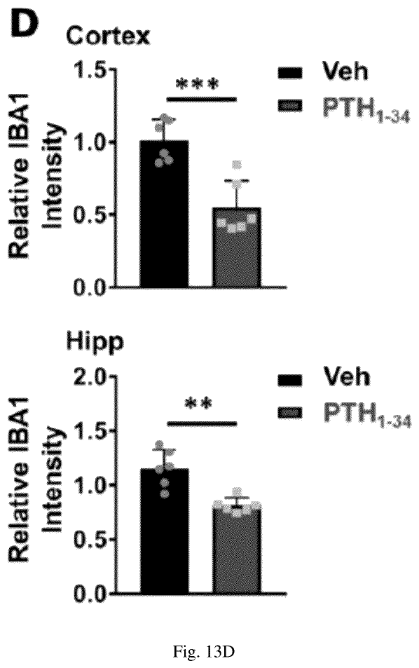

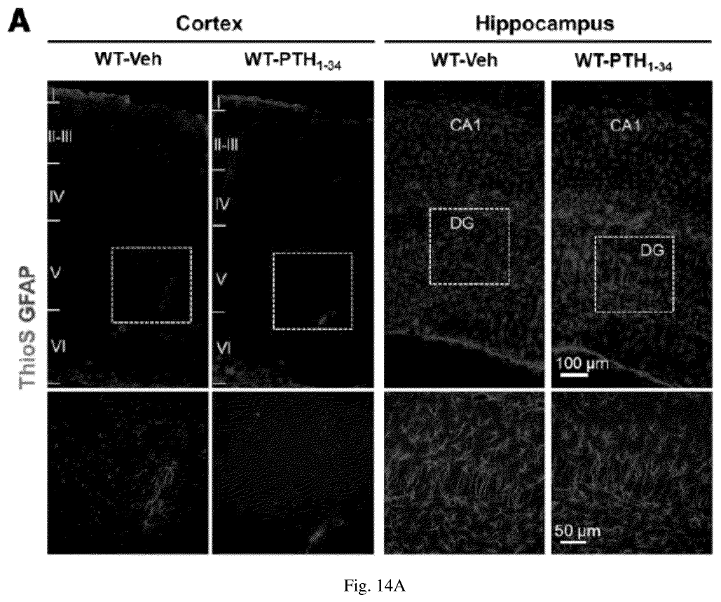

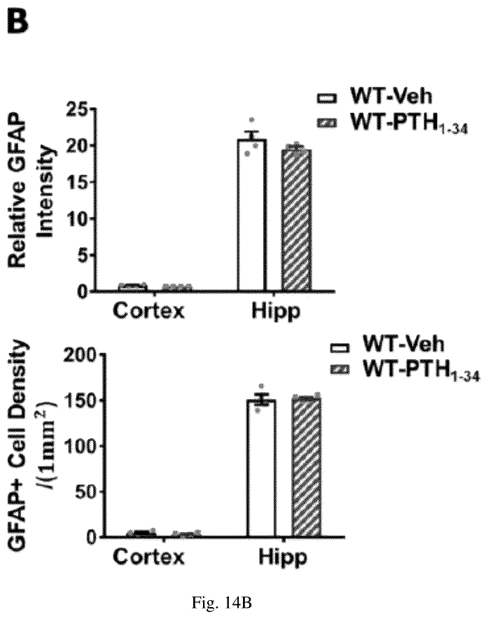

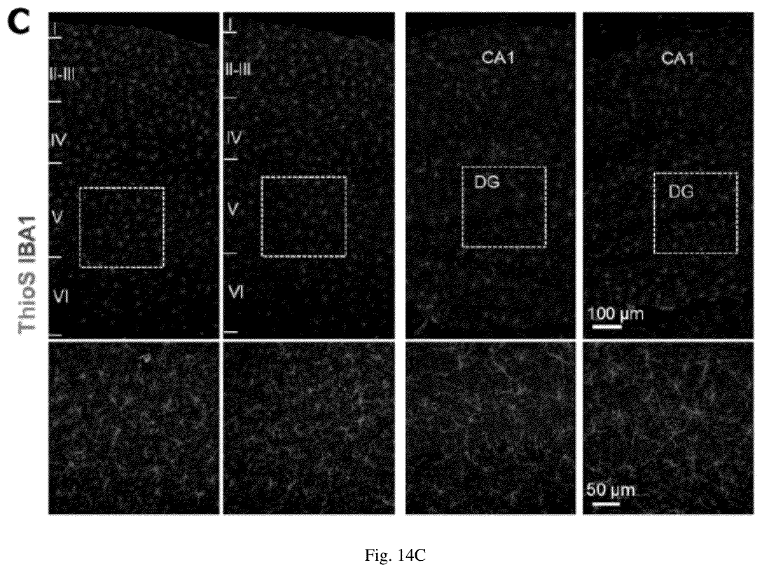

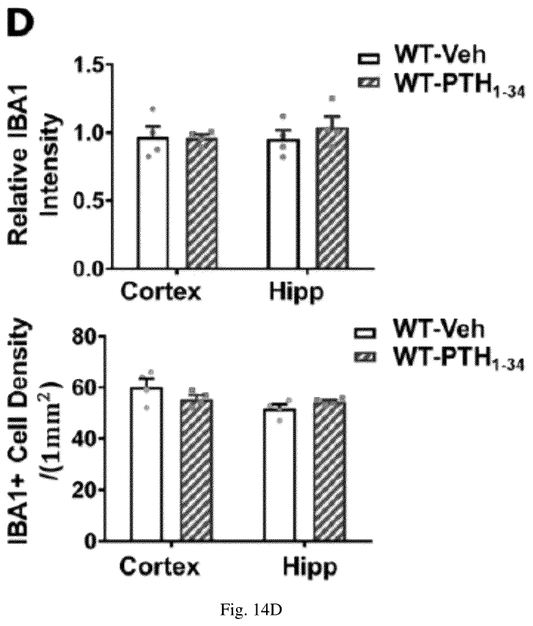

To further identify the effect of PTH₁₋₃₄ on Aβ plaque-associated pathology, we examined GFAP+ astrocytes in female 5×FAD brains with PTH₁₋₃₄ or Veh treatments (˜6 MO). As shown in FIG. 4A-B, co-immunostaining analysis of GFAP (a marker for reactive astrocytes in cortex) with ThioS showed a similar overall response as those of IBA1+ microglial cells to PTH₁₋₃₄ treatments. The increase in GFAP+ reactive astrocytes was found in areas of Aβ deposition in 5×FAD-Veh mice, including each layer of the cortex, as well as the DG region of the hippocampus. The GFAP+ fluorescence intensity in layers of cortex (except layer II/III) and DG region of hippocampus were lower in PTH₁₋₃₄ treated 5×FAD mice than that of Veh controls (FIG. 4A-B), in correlation with the reduced Aβ plaques by PTH₁₋₃₄ treatments. However, in contrast to unchanged AP-plaque associated IBA1/LPL fluorescence (FIG. 12D-G), the plaque-associated GFAP fluorescence appeared to have diminished by PTH₁₋₃₄ treatments (FIG. 4C-D). There were significant numbers of ThioS+plaques with little or no GFAP+ astrocytes surrounding the brain (cortex and hippocampus) of PTH₁₋₃₄ treated 5×FAD mice (FIG. 4C). As the overall fluorescence intensity of IBA1 and GFAP decreased in PTH₁₋₃₄ treated 5×FAD brain, we further tested this view by Western blot analysis. Certainly, lower IBA1 and GFAP protein levels were detected in PTH₁₋₃₄ treated 5×FAD brain homogenates (cortex and hippocampus) (FIG. 4E-F), supporting the view for an overall reduction in glial reactivation by PTH₁₋₃₄. Notice that this PTH₁₋₃₄ inhibition of glial cell activation was detectable in both female and male 5×FAD mice (FIGS. 4, 13); but PTH₁₋₃₄ had little effect in these glial cells in wild-type (WT) mice (FIG. 14). These results thus suggest PTH₁₋₃₄'s inhibitory effect on both astrocyte and microglial activation in 5×FAD, but not WT, mice in a sex-independent manner.

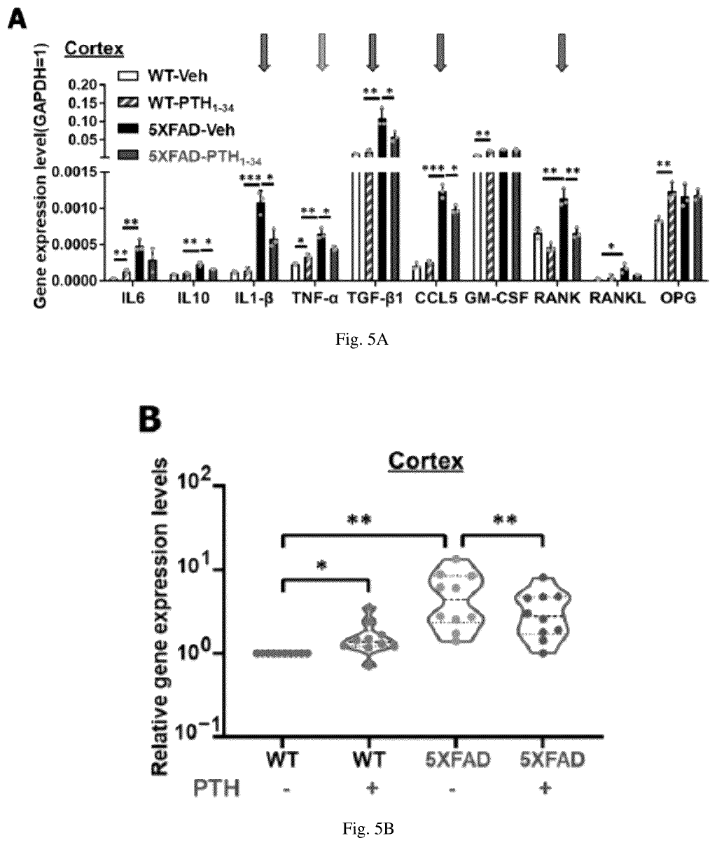

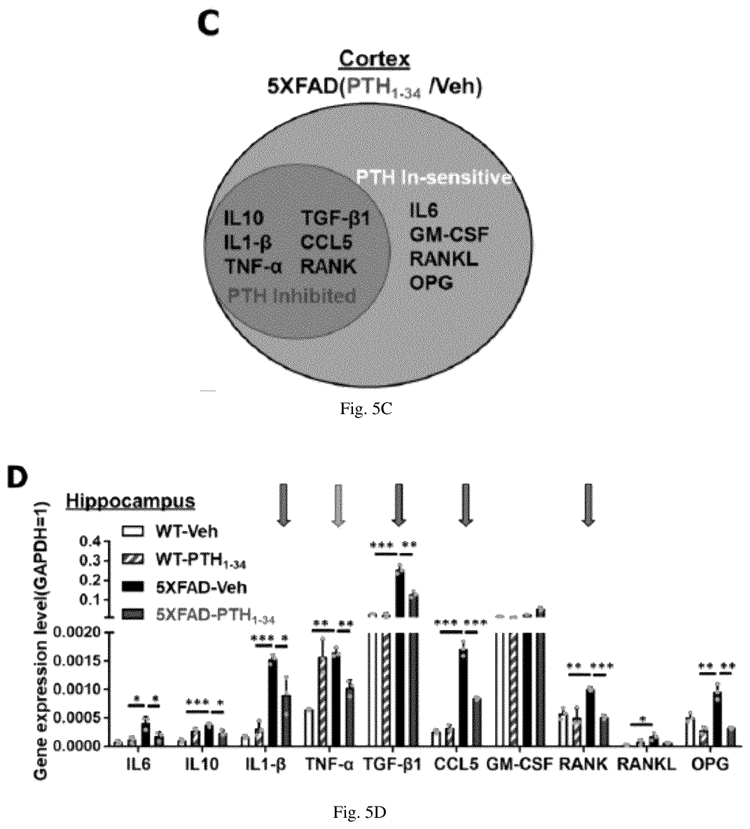

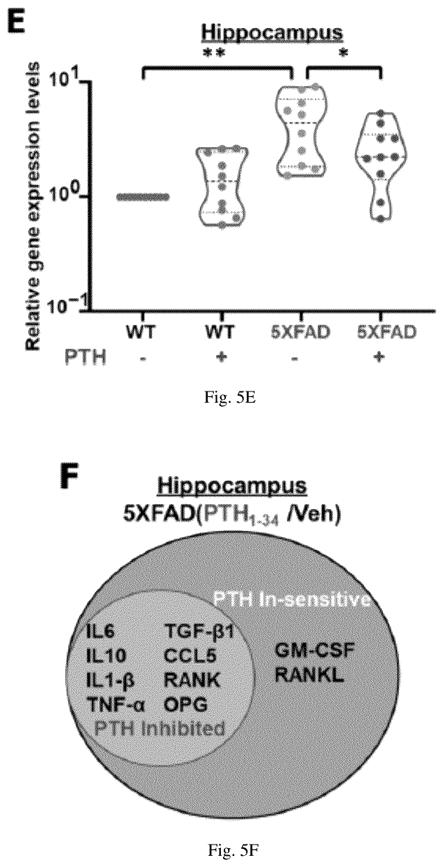

We then asked whether PTH₁₋₃₄ treatments affected the expressions of proinflammatory cytokines and chemokines in 5×FAD brain. RT-qPCR analyzed ˜10 genes' expressions, which are either well-recognized proinflammatory cytokines (e.g., IL1β, IL6, TNFα, TGFβ) or cytokines involved in bone remodeling (e.g., RANKL, RANK, OPG, and GM-CSF), in 6˜MO old WT/5×FAD female brain (cortex and hippocampus) with PTH₁₋₃₄ or vehicle treatments (FIG. 5A, D). While most of these factors were upregulated in 5×FAD brain (cortex and hippocampus), as compared with those of same-aged WT mice, these upregulated inflammatory cytokines or chemokines were largely brought down to nearly normal levels by PTH₁₋₃₄ (FIG. 5A-B, D-E). Notice that while the majority of factors were downregulated by PTH₁₋₃₄ treatments in both 5×FAD cortex and hippocampus, a few cytokines, largely factors involved in bone remodeling (e.g., GM-CSF, RANKL, and OPG in the cortex) occurred unaffected by PTH₁₋₃₄ (FIG. 5C, F). This is in contrast to the effect of PTH in osteoblastic bone cells, where PTH is known to regulate RANKL and OPG expression. Taken together, these results suggest that PTH₁₋₃₄ treatments reduce glial cell activation and consequent brain inflammation.

### PTH₁₋₃₄ Reduction of Serum Inflammatory Cytokines

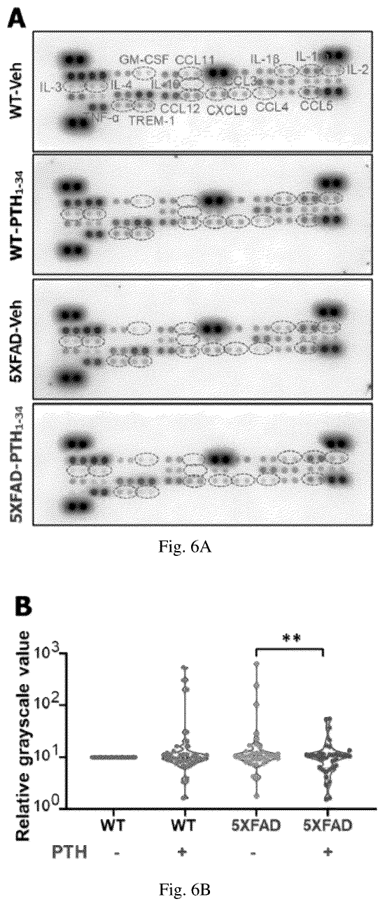

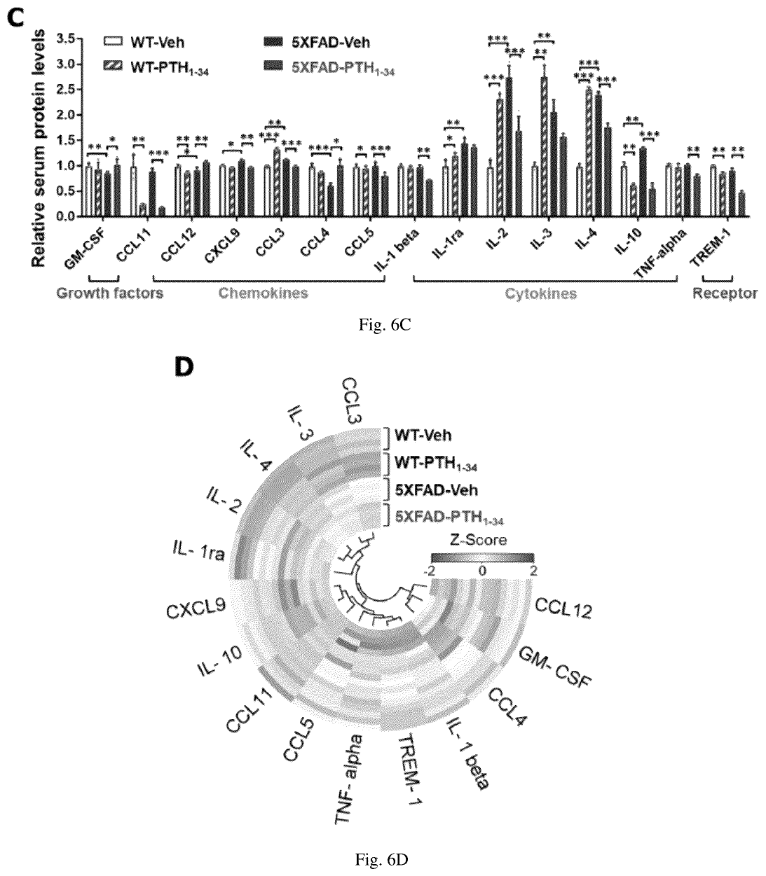

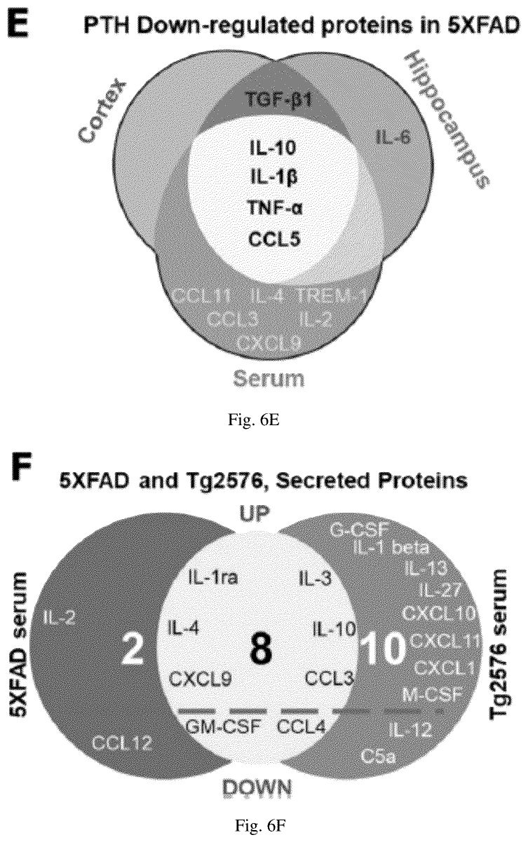

To understand how PTH₁₋₃₄ treatments affect brain inflammation in 5×FAD mice, we investigated PTH₁₋₃₄'s effect on systemic inflammation, since PTH is a hormone with systemic effects, particularly in bone cells. Using multiplexed antibody-based arrays to screen for altered serum/plasma proteins in 5×FAD and WT female mice (6˜MO) with PTH₁₋₃₄ or vehicle treatments, we found that among 40 factors, few (7/40) were elevated in serum samples from 5×FAD mice, as compared with that of WT mice (FIG. 6A-C), suggesting a weak systemic inflammation in 5×FAD mice. Notice that most of these elevated serum factors (e.g., IL-1rα, IL-2, IL-3, IL-4, IL-10, CXCL9, CCL3) in 5×FAD mice were all reduced by PTH₁₋₃₄ treatments (FIG. 6C-D). Additionally, PTH₁₋₃₄ reduced some serum inflammation-associated factors such as IL1β, TNFα, TREM-1, CCL11, and CCL5, which were comparable between WT and 5×FAD mice, and restored serum factor CCL4, which was decreased in 5×FAD mice (FIG. 6C-D). We compared the PTH₁₋₃₄ downregulated cytokines in serum of 5×FAD mice with those in brain of 5×FAD, as shown in FIG. 6E. The results showed that 4 factors, IL-10, IL-1β, TNFα, and CCL5, were reduced by PTH₁₋₃₄ treatments not only in the serum but also in the brain of 5×FAD, suggesting a certain relationship between systemic inflammation and brain inflammation (FIG. 6E). We further compared these changes in serum factors between 5×FAD and Tg2576 mice, another AD animal model expressing Swedish mutant APP ubiquitously. In contrast to that of 5×FAD mice, Tg2576 mice exhibited elevations in many (15/40) of these serum factors (FIG. 6F). These results demonstrate the systemic effect of PTH₁₋₃₄, although systemic inflammation in 5×FAD mice seems to be weaker than in Tg2576 mice.

PTH₁₋₃₄ Association with Astrocytes in the Brain

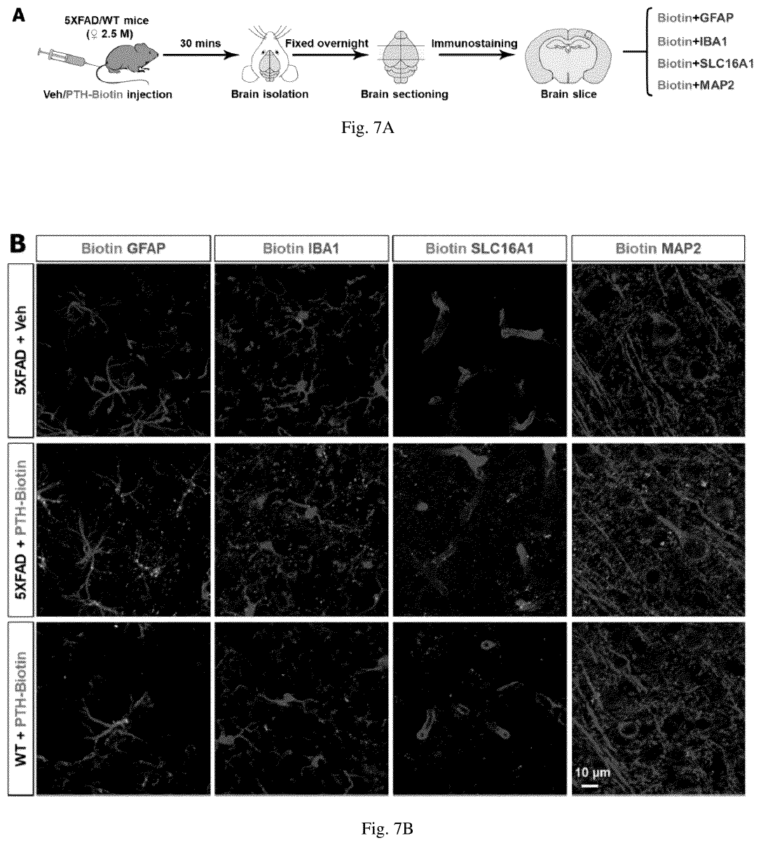

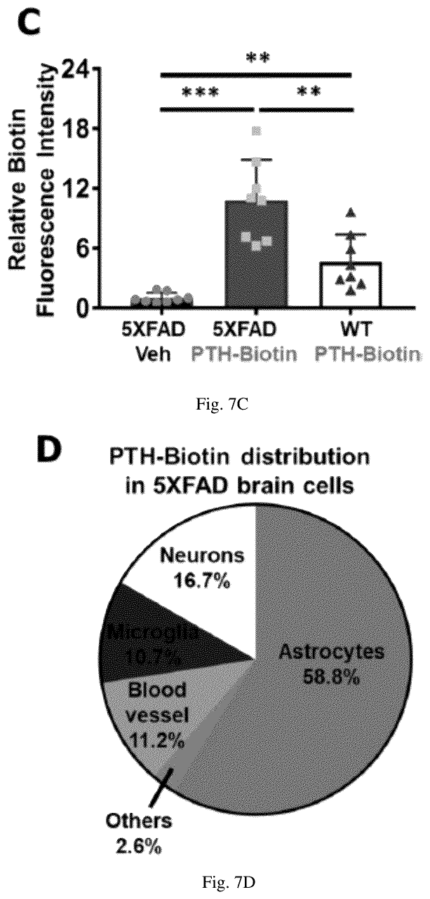

Given the significant effect of PTH₁₋₃₄ in 5×FAD brain pathology, we wondered whether the injected PTH₁₋₃₄ functioned directly in 5×FAD brain. To address this question, Biotin-conjugated PTH₁₋₃₄ was administered into WT and 5×FAD female mice (˜2.5 MO) by caudal vein injection, and mice were sacrificed 30 min after injection, as illustrated in FIG. 7A. Immunostaining analysis with biotin antibody showed more biotin signals in the brain of 5×FAD mice than in WT mice (FIG. 7B-C), implicating a BBB (blood-brain barrier) leakage in 5×FAD brain that causes more Biotin PTH1-34 to enter the brain. Further co-immunostaining analysis showed that most Biotin-PTH₁₋₃₄ signals were associated with GFAP+ astrocytes (˜60%); ˜10-17% of Biotin-PTH₁₋₃₄ signals were in association with IBA1+ microglia, SLC16A1+ blood vessels, and MAP2+ neurons, respectively (FIG. 7D). These results thus suggest that injected biotin-PTH₁₋₃₄ can enter the brain through BBBs of 5×FAD mice and bind to astrocytes in large quantities.

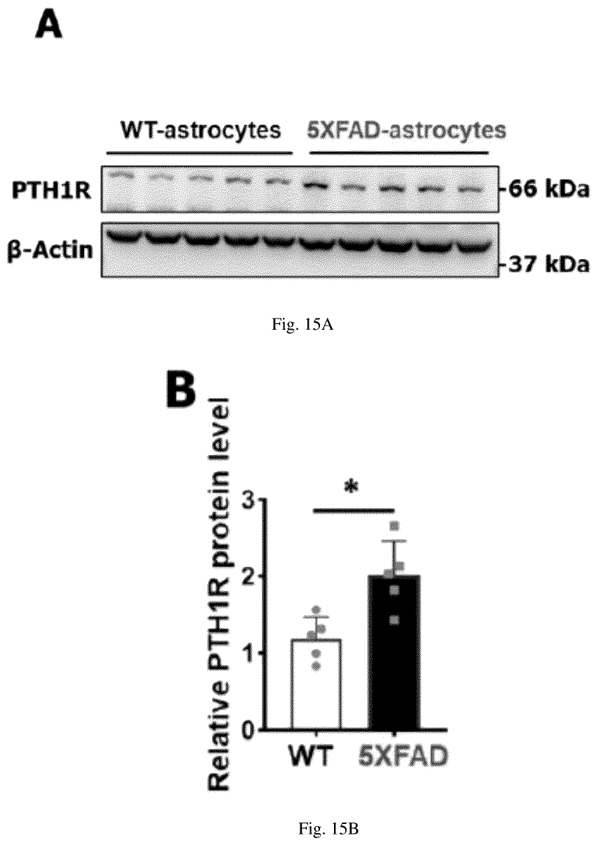

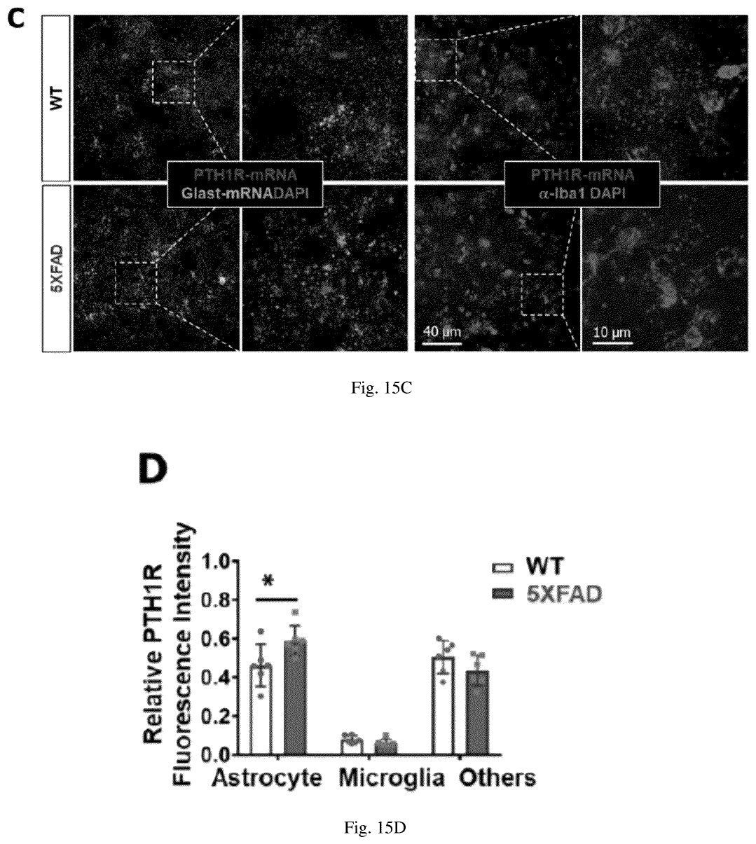

The abundant association of Biotin-PTH₁₋₃₄ with astrocytes leads us to ask whether this is due to the abundant expression of PTH receptor (PTH1R) in astrocytes. Indeed, scRNA-seq database showed selective PTH1R expression in astrocytes and endothelial cells in the brain. We further verified this view by RNA scope analysis (a higher resolution of in situ analysis), which showed co-distribution of PTH1R's mRNAs largely with Glast1-mRNA+ astrocytes (FIG. 15C-D). Moreover, western blot analysis of lysates from primary cultured astrocytes demonstrated expression of PTH1R (FIG. 15A B). Interestingly, the PTH1R protein level appeared to be slightly higher in astrocytes from 5×FAD mice than that of WT astrocytes (FIG. 15B-D). Together, these results support the view that injected PTH₁₋₃₄ can travel to the brain and bind to its receptor in astrocytes in 5×FAD mice.

### PTH₁₋₃₄ Suppression of Proinflammatory Cytokines' Expression in 5×FAD Astrocytes

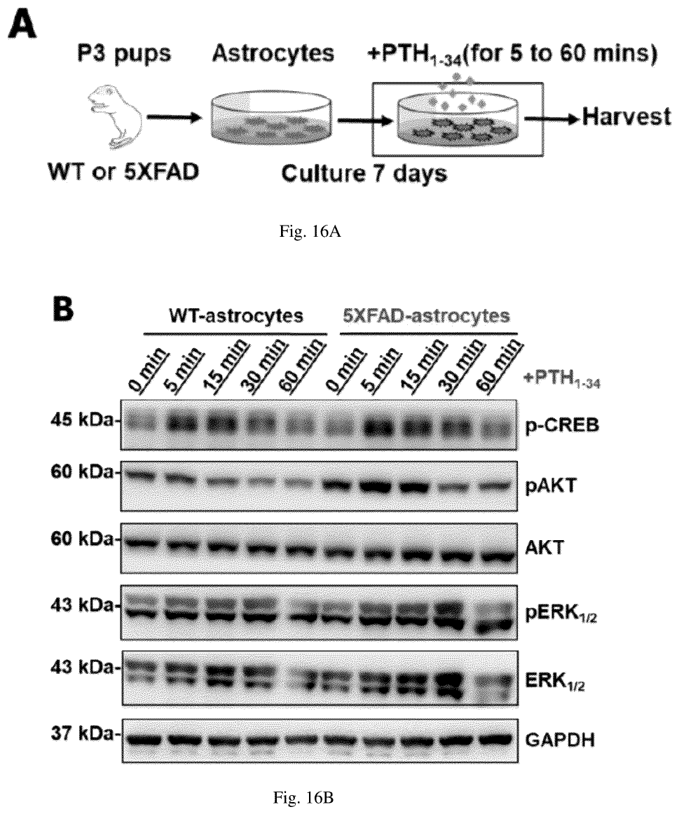

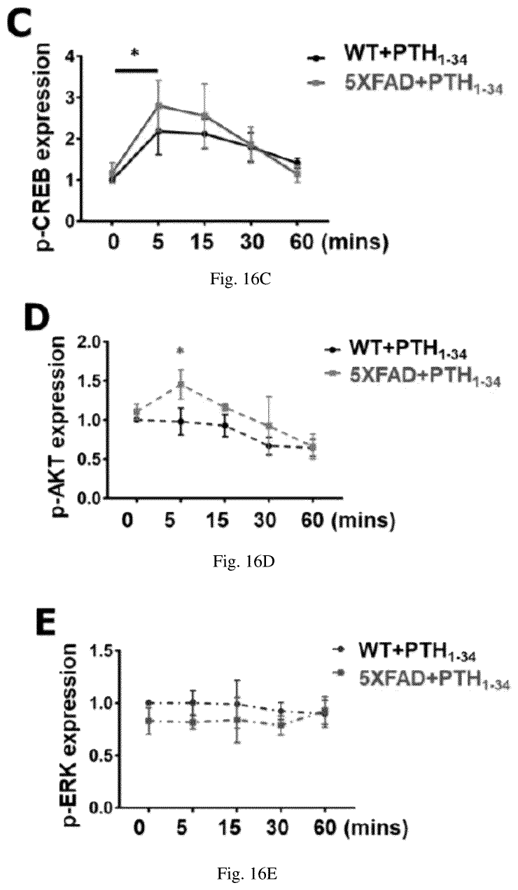

To verify the view that PTH₁₋₃₄ functions directly on astrocytes, we examined whether PTH₁₋₃₄ could induce signaling and function in primary cultured astrocytes from WT and 5×FAD female pups (FIG. 16A). Interestingly, among PTH₁₋₃₄ induced signaling pathways identified in osteoblasts (e.g., cAMP-driven phospho-CREB, phospho-AKT, and phospho-ERK1/2), PTH₁₋₃₄ increases p-CREB, but has little effect on either p-AKT or p-ERK (FIG. 16B-E), suggesting that PTH₁₋₃₄ induction of cAMP to p-CREB may be a key signaling pathway in astrocytes. Notice that a trend of increasing, but not significant, p-CREB was detected in 5×FAD astrocytes, as compared to WT astrocytes (FIG. 16C), which might be due to a slight increase in the expression of PTH1R in 5×FAD astrocytes. These results thus support the view that PTH₁₋₃₄ functions directly on astrocytes.

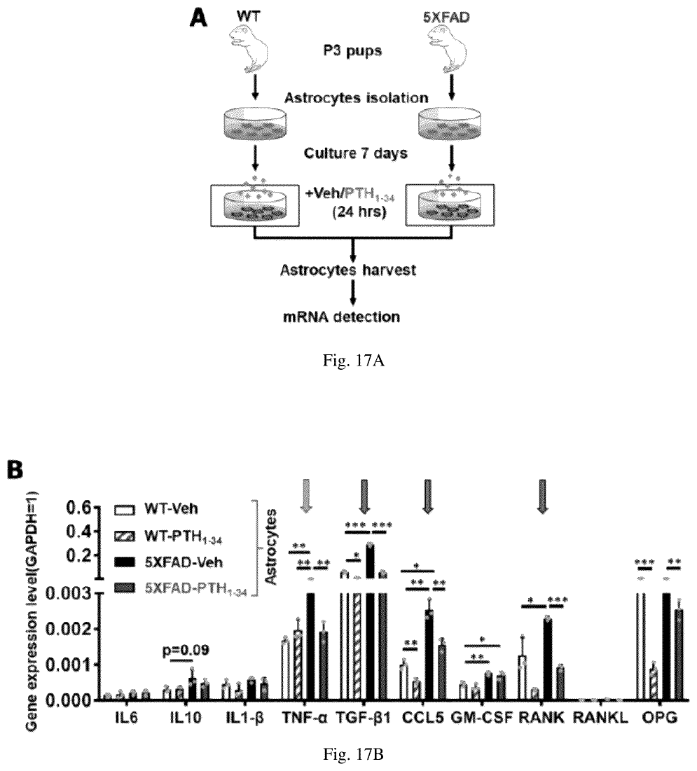

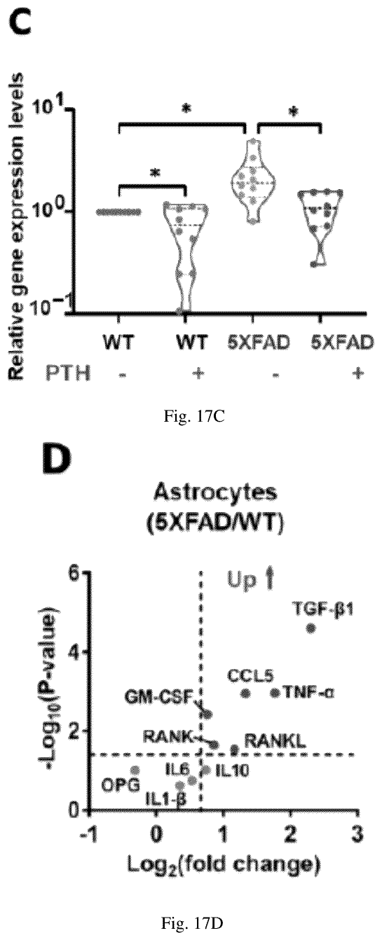

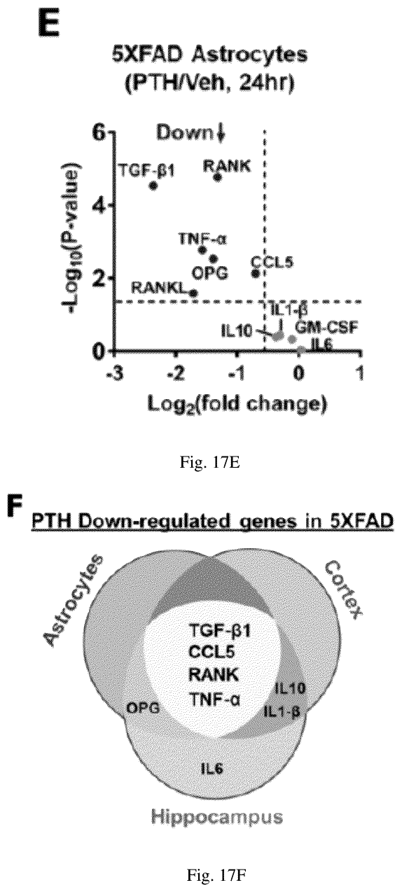

We further tested this view by examining PTH₁₋₃₄'s effect on the expression of cytokines and chemokines in astrocytes. Cultured astrocytes from WT and 5×FAD female pups were treated with vehicle or PTH₁₋₃₄ for 24 h, and then subjected to RT-qPCR analysis, as illustrated in FIG. 17A. Increased levels of cytokines or chemokines (e.g., TNF-α, TGF-β1, CCL5, GM-CSF, and RANK) were detected in 5×FAD astrocytes, as compared with those in WT astrocytes (FIG. 17B-D), consistent with multiple literature reports of a higher basal inflammatory state in 5×FAD cells. Nearly all these factors (except GM CSF) were reduced by PTH1-34 treatments (FIG. 17B, E). We compared these changes with PTH1-34 downregulated genes in the 5×FAD brain (cortex and hippocampus). As illustrated in FIG. 17F, 4 factors, TGF-β1, CCL5, RANK, and TNFα, were reduced by PTH₁₋₃₄ treatments not only in cultured 5×FAD astrocytes but also in 5×FAD brain. These results provide additional support for PTH₁₋₃₄ functioning in astrocytes, where it suppresses the expression of inflammatory cytokines or chemokines.

PTH₁₋₃₄ Decrement of Senescence-Like Astrocytes from 5×FAD Mice

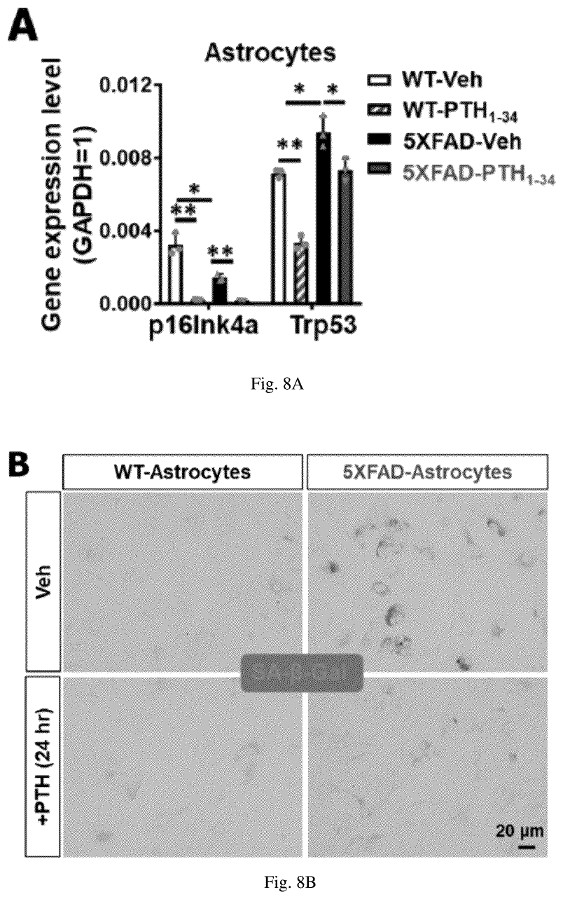

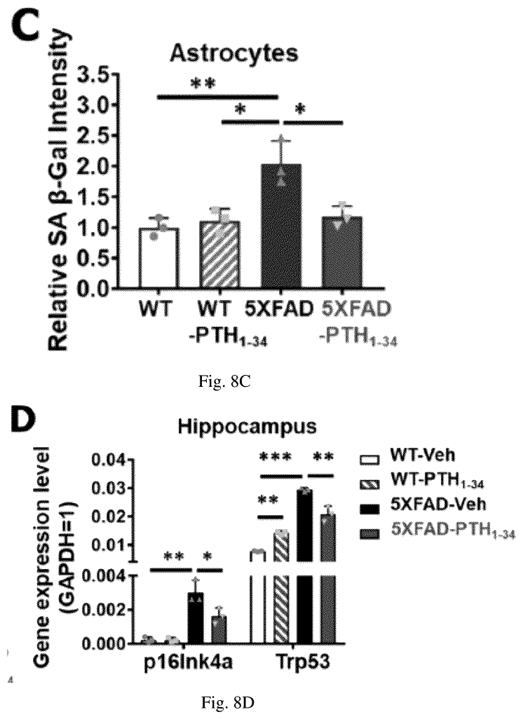

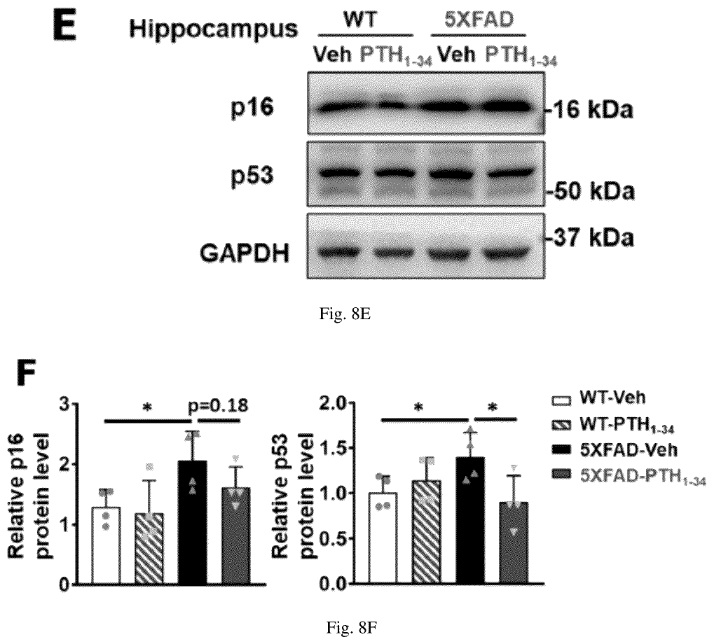

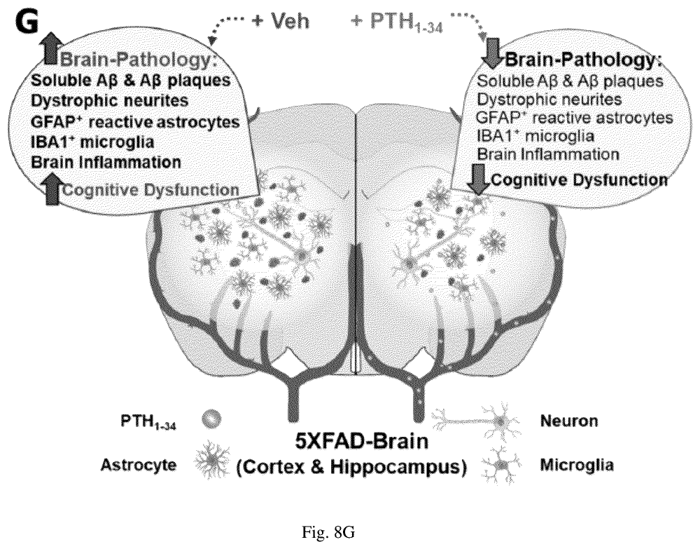

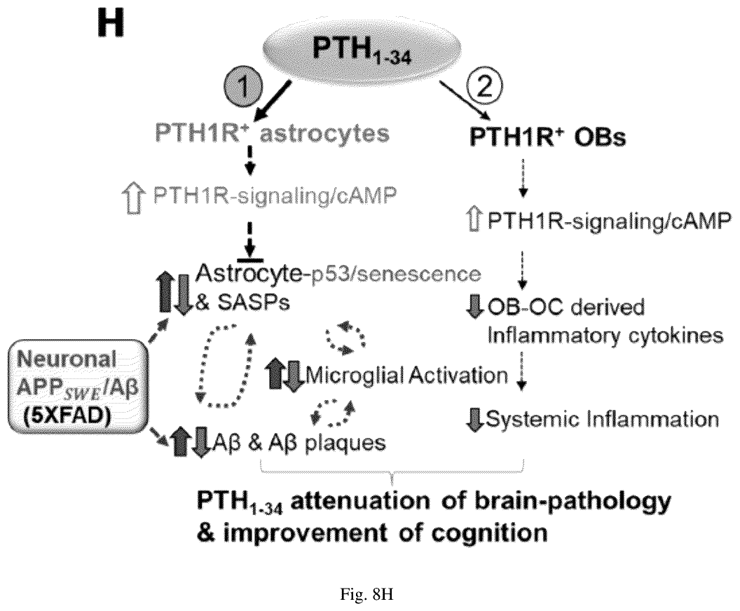

It is known that increased proinflammatory cytokines and chemokines in the brain/astrocytes of 5×FAD mice exhibit features of SASPs (senescence-associated secretory phenotypes). Given reports of astrocyte senescence in the brains of AD patients, we asked whether astrocytes from 5×FAD mice showed cellular senescence features and whether this event was affected by PTH₁₋₃₄ treatments. To this end, cultured astrocytes from WT and 5×FAD female pups were treated with PTH₁₋₃₄ or vehicle for 24 h and examined the expression of senescence markers, including p16Ink4a, p53, and SA-β-gal (senescence associated β-gal). RT-PCR analysis showed increased p53, but not p16Ink4a, in 5×FAD astrocytes, which was diminished by PTH₁₋₃₄ (FIG. 8A), implicating possible senescence in 5×FAD astrocytes. This view was further supported by SA-β-gal analysis, which showed increased SA-3-Gal+ astrocytes from 5×FAD mice, and attenuated by PTH₁₋₃₄ treatments (FIG. 8B-C). Given the above data, we subsequently tested this view in vivo. RT-PCR analysis of both p16Ink4a and p53 transcripts showed a clear increase in the 5×FAD hippocampus and a great reduction after PTH₁₋₃₄ treatment, compared with WT group (FIG. 8D). In line with these results, western blot analysis showed an increase in p16Ink4a and p53 in the female 5×FAD hippocampus homogenates, with p53 reduced by PTH₁₋₃₄ treatments (FIG. 8E-F). Together, these results suggest that p53 may be a driver of astrocyte senescence in the brain of 5×FAD mice, which is attenuated by PTH₁₋₃₄, implicating PTH₁₋₃₄ decrement of astrocyte senescence in 5×FAD mice as a potential cellular mechanism for its beneficial effects.

PTH₁₋₃₄ intermittent treatments not only increased trabecular bone volumes and bone formation (FIG. 1) but also decelerated AP-associated brain pathology in 5×FAD mice (FIG. 8G). The Aβ levels, Aβ deposition (FIG. 3), dystrophic neurites (FIG. 11), reactive microglia and astrocytes (FIG. 12A-B and FIG. 4), and brain inflammatory (FIG. 5) were all diminished by PTH₁₋₃₄ treatments in 5×FAD mice, and the cognitive functions (FIG. 2) in 5×FAD mice (in particular, female) were also improved. Notably, the therapeutic effect of PTH₁₋₃₄ was enhanced in female mice, as amyloid deposition levels in 5×FAD female mice after PTH₁₋₃₄ treatment were reduced by a greater proportion than in male mice (FIG. 3D-L). PTH₁₋₃₄ treatment brings the level of AB deposition in female 5×FAD mice down to the level of untreated 5×FAD male mice or even lower. Such a female-dependent PTH₁₋₃₄ effect may be due to the earlier and more severe onset of amyloid pathology in female 5×FAD mice.

As illustrated in FIG. 8H, we speculate that PTH₁₋₃₄, by binding to its receptor PTH1R in bone osteoblasts and brain astrocytes, suppresses cellular senescence, systemic inflammation, and brain inflammation, thereby alleviating AD pathology in 5×FAD mice. This speculation is in light of the following observations. First, PTH1R is expressed not only in osteoblasts but also in astrocytes (FIG. 15). Second, PTH₁₋₃₄ may affect cytokine expression not only in osteoblasts (e.g., RANKL, RANK, OPG, TGF-01, IL-6, TNF-α, etc.), but also in astrocytes (FIG. 17). Third, PTH₁₋₃₄ appears to act as a senolytic drug, with a recent report showing that PTH protects bone from age-induced bone loss by protecting osteoblasts/osteocytes from oxidative stress-induced cell death and senescence. PTH₁₋₃₄ suppressed astrocyte senescence (FIG. 8A-C), an event that induces SASPs and inflammatory cytokine expression. Although PTHrP (Parathyroid hormone-related protein) has been reported in neurons in normal mouse brains and can be induced in reactive astrocytes in inflamed brains or after brain injury, our study shows that exogenous PTH₁₋₃₄ can enter the brain and bind to astrocytes. It is important to note that the Biotin-PTH₁₋₃₄ signals were detectable in the brains of WT mice after its injection, suggesting the possibility of “PTH₁₋₃₄ infiltration into the brain” of WT mice under normal BBB condition. However, more Biotin PTH₁₋₃₄ enters the brain of 5×FAD mice, as compared to that of WT controls (FIG. 7B-C). This may be due to the BBB impairment in 5×FAD mice. It is noteworthy that the expression of PTH1R in brain has been detected in other species, including zebrafish, chicken, or xenopus. We believe that astrocytes may be the dominant cell type in which PTH₁₋₃₄ functions in the brain, as the majority of injected biotin-PTH₁₋₃₄, is associated with astrocytes (FIG. 7D); astrocytes express abundant PTH1R (FIG. 15), and PTH1-34 could induce signaling in primary cultured astrocytes (FIG. 16).

PTH₁₋₃₄ appears to be a senolytic-like drug that not only protects osteoblasts/osteocytes from oxidative stress-induced cell death and senescence, but also reduces the senescence phenotype in astrocytes (FIG. 8A-C). We detected increased senescence markers (e.g., SA-β-gal, and 53) in cultured primary astrocytes from 5×FAD mice. Upon PTH₁₋₃₄ treatments, these senescence markers in astrocytes were reduced, as were SASP-like cytokines in 5×FAD astrocytes and 5×FAD brain (FIG. 8A-C). These observations thus support the view for PTH₁₋₃₄ to act as a senolytic-like drug.

Our results of decreased total GFAP+ astrocytes (FIG. 4) and brain inflammatory cytokines (FIG. 5) in PTH₁₋₃₄ treated 5×FAD mice also suggest a role of PTH₁₋₃₄ in suppressing astrocyte activation. This view is also supported by the results obtained in cultured astrocytes, which showed downregulation of cytokines and chemokines (TGF-β1, TNF-α, CCL5, RANK) in 5×FAD astrocytes treated with PTH₁₋₃₄ (FIG. 17).

In addition to its function in astrocytes, PTH₁₋₃₄'s effect on osteoblasts and systemic inflammation may further underlie its beneficial roles in inhibiting AD-related brain pathology. Evidence suggests that systemic inflammation may influence local inflammation in the diseased brain, leading to excessive synthesis of inflammatory cytokines and other mediators in the brain, which in turn may contribute to the outcome or progression of chronic neurodegenerative disease. Chronic inflammation is a common risk factor for both osteoporosis and AD. Systemic inflammation, including a few elevated serum inflammatory factors, was nearly abolished by PTH₁₋₃₄ treatments in 5×FAD mice (FIG. 6A-D), suggesting a systemic effect of PTH₁₋₃₄. However, a comparison of serum inflammation factors between the 5×FAD and Tg2576 mice showed a weaker systemic inflammation in 5×FAD mice than that in the Tg2576 AD animal model (FIG. 6F). This may be due to the differential mutant APP expression pattern between the two AD mouse lines. While Tg2576 mice express mutant APP ubiquitously, including in bone cells, under the control of prion promoter, 5×FAD mice express mutant APP under the control of Thy1 promoter, thus driving the expression of mutant APP largely in neurons and immune cells.

In summary, this example describes the anti-senile function of PTH₁₋₃₄ intermittent treatments in 5×FAD mice, exhibiting PTH₁₋₃₄ induced reductions in Aβ pathology, systemic and brain inflammation, and PTH₁₋₃₄ improvement in cognitive function in 5×FAD mice and implicating PTH₁₋₃₄'s potential therapeutic effects for patients with not only osteoporosis but also AD. At cellular level, PTH₁₋₃₄ binds to PTH1R not only in osteoblasts but also in astrocytes, suppressing cellular senescence, systemic inflammation, and brain inflammation.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, the following is claimed:

1. A method of treating Alzheimer's disease (AD) in a subject comprising administering to the subject a therapeutically effective amount of at least one parathyroid hormone type 1 receptor (PTH1R) agonist.

2. The method of claim 1, wherein the PTH1R agonist is administered at an amount effective to increase cognitive function and/or inhibit one or more AD brain pathologies in the subject.

3. The method of claim 2, wherein the one or more AD brain pathologies are selected from the group consisting of astrocyte senescence, glial cell activation, expression of brain inflammatory cytokines, brain inflammation, systemic inflammation, dystrophic neurites, Aβ accumulation and Aβ deposition.

4. The method of claim 1, wherein the PTH1R agonist is administered at an amount effective to increase bone mass, bone density, bone thickness, and bone formation, or decrease bone resorption in the subject.

5. The method of claim 1, wherein the PTH1R agonist is intermittently administered to the subject.

6. The method of claim 1, wherein the PTHR1 agonist comprises parathyroid hormone (PTH), or a PTH analog.

7. The method of claim 6, wherein the PTH analog comprises a PTH₁₋₃₄ peptide.

8. The method of claim 1, further comprising measuring the bone mineral density (BMD) or bone thickness of the subject's skull and administering the PTH1R agonist to the subject if the measured BMD or bone thickness of the subject's skull is reduced or decreased compared to a control BMD or bone thickness.

9. The method of claim 7, wherein the BMD or bone thickness of the subject's skull is measured using a computed tomography (CT) or X-ray modality.

10. The method of claim 1, wherein the PTH1R agonist is administered subcutaneously to the subject.

11. A method of treating AD in a subject comprising:
measuring BMD or bone thickness of the skull of the subject; and
administering to the subject a therapeutically effective amount of at least one PTH1R agonist if the measured BMD or bone thickness of the subject's skull is reduced or decreased compared to a control BMD or bone thickness.

12. The method of claim 11, wherein the therapeutically effective amount is the amount effective to increase cognitive function or inhibit one or more AD brain pathologies in the subject.

13. The method of claim 12, wherein the one or more AD brain pathologies are selected from the group consisting of astrocyte senescence, astrocyte activation, expression of brain inflammatory cytokines, brain inflammation, systemic inflammation, dystrophic neurites, Aβ accumulation and Aβ deposition.

14. The method of claim 11, wherein the therapeutically effective amount is the amount effective to increase one or more of bone mass, bone density, bone thickness, bone formation, and bone resorption in the subject.

15. The method of claim 11, wherein the PTH1R agonist is intermittently administered to the subject.

16. The method of claim 11, wherein the PTHR1 agonist comprises parathyroid hormone (PTH), or a PTH analog.

17. The method of claim 16, wherein the PTH analog comprises a PTH₁₋₃₄ peptide.

18. The method of claim 11, wherein the PTH1R agonist is administered subcutaneously to the subject.

19. The method of claim 11, wherein the BMD or bone thickness of the subject's skull is measured using a computed tomography (CT) or X-ray modality.

20. A method of diagnosing Alzheimer's disease in a subject, the method comprising measuring the bone marrow density (BMD) or bone thickness of a subject's skull, wherein a reduced BMD or bone thickness compared to a control BMD or bone thickness is indicative of the subject having Alzheimer's disease or having an increased risk of Alzheimer's disease.
